(19) United States
(12) Patent Application Publication
(10) Pub. No.: US 2021/0123932 A1
(43) Pub. Date: Apr. 29, 2021

(54) Genetic Determination of Hormone Levels and Applications Thereof

(71) Applicant: The Board of Trustees of the Leland Stanford Junior University, Stanford, CA (US)

(72) Inventors: Manuel A. Rivas, Palo Alto, CA (US); Emily Flynn, Stanford, CA (US); Yosuke Tanigawa, Stanford, CA (US)

(73) Assignee: The Board of Trustees of the Leland Stanford Junior University, Stanford, CA (US)

(21) Appl. No.: 17/079,110

(22) Filed: Oct. 23, 2020

Related U.S. Application Data
(60) Provisional application No. 62/925,133, filed on Oct. 23, 2019.

(51) Int. Cl. G01N 33/74; C12Q 1/6869; A61K 38/22

(52) U.S. Cl. CPC G01N 33/743; A61K 45/06; A61K 38/22; C12Q 1/6869

(57) ABSTRACT

Methods to determine hormone level of an individual and applications thereof are described. Generally, systems utilize genetic variants to determine hormone level, which can be used as a basis to inform health status and treat individuals.

### CROSS REFERENCE TO RELATED APPLICATIONS

This application claims priority to U.S. Provisional Application Ser. No. 62/925,133, entitled “Genetic Determination of Hormone Levels and Applications Thereof,” to Rivas et al., filed Oct. 23, 2019, which is incorporated herein by reference in its entirety.

### STATEMENT REGARDING FEDERALLY SPONSORED RESEARCH OR DEVELOPMENT

This invention was made with Government support under contract F31 LM013053 awarded by the National Institutes of Health. The Government has certain rights in the invention.

### TECHNICAL FIELD

The disclosure is generally directed toward methods and systems to assess hormone level utilizing genetic data and various applications thereof.

### BACKGROUND

Hormones are a class of biological molecules that provide signal throughout the body to regulate physiology and behavior. Hormones communicate between organs and tissues for physiological regulation and behavioral activities including metabolism, growth, development, reproduction, fertility, appetite, respiration, tissue function, sensory perception, sleep, movement and mood. Hormones may be secreted into the bloodstream from a number of tissues, especially the hypothalamus, pituitary gland, thyroid, adrenal gland, and gonads.

Testosterone is a hormone with differential biological effects on men and women. Importantly, it regulates body fat composition, libido, and bone density. Problems with testosterone (over or underproduction) are a common cause of unexplained infertility. Women also have low levels of circulating testosterone, largely produced by the adrenal glands and to a lesser extent by the ovaries. High levels of testosterone are associated with insulin resistance, dyslipidemia, hypertension, polycystic ovarian syndrome (PCOS) disease, virilization, amenorrhea, and hirsutism. Testosterone supplementation is widely prescribed in men for a variety of reasons, but it's secondary effect on cardiovascular disease and other risk factors remains important to consider.

### SUMMARY

Various embodiments are directed towards systems and methods for determining hormone levels from genetic data. In various embodiments, an individual's hormone level is determined utilizing a linear model and the individual's genetic variants. In various embodiments, a linear model is specific for particular sex. In various embodiments, a hormone level assessment is utilized to provide a medically related intervention and/or treat the individual accordingly.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1 provides a flow chart of a method to determine hormone levels of an individual utilizing genetic data and linear model, in accordance with various embodiments.

FIG. 2 provides Table 1 on SEMM genetic parameter estimates for anthropometric traits, utilized in accordance with various embodiments. For each trait (trait, UK Biobank data field ID [UKBB ID], parameter fits (pi[0] is the null and pi[1] is the non-null fraction, and Sigma is the variance-covariance matrix) and derived quantities, including heritability (hf=female-specific heritability, hm=male-specific heritability) and between-sex genetic correlation (rg); 2.5 to 97.5 percentile intervals are given for each value with/suffix indicating the lower limit and u suffix indicating the upper limit.

FIG. 3 provides Table 2 on quantitative traits examined, utilized in accordance with various embodiments. Biomarker traits are listed with their UK Biobank Field ID (UKB ID).

FIG. 4 provides Table 3 on traits included in Mendelian Randomization analysis, utilized in accordance with various embodiments. Contains the trait, the source of the trait, and the MRBase ID. The table also includes the sample_size of the population and whether the population is single sex or both sexes combined.

FIG. 5 provides a schematic overview of the sex-effect mixture model (SEMM), in accordance with various embodiments. A dataset of 33 serum and urine biomarkers from 358,072 individuals in UK Biobank was analyzed. GWAS summary statistics was calculated from males (light purple) and females (orange) separately, so that for every trait, we had an effect estimate (\hat{β}_{f} and \hat{β}ₘ) and standard error for each variant in each sex. A two-component Bayesian Sex-Effect Mixture Model (SEMM) was used, with no effect and non-zero effect components, to estimate SNP-based heritability and genetic correlation between males and females for each biomarker. A four-component extension of the SEMM contains two additional components for separate male and female effects. This model distinguishes between four cases: genetic variants that have no effect (illustrated as M0), genetic variants that have a stronger association with the trait in females or males (M1, orange, or M2, light purple), and genetic variants that have similar effects in females and males (M3, gray). SEQ ID Nos. 1 to 6.

FIGS. 6A and 6B provide comparison of estimates for anthropomorphic traits, utilized in accordance with various embodiments. 6A) Genetic correlation estimates from SEMM (purple). The 2.5 to 97.5 percentile interval from STAN sampling shown for our estimates, 95% CI shown for all anthropometric traits except fat-ratio genetic correlations, which are unavailable. 6B) Heritability estimates (circles) for males (light purple) and females (orange). Error bars show the 2.5 to 97.5 percentile interval (for our estimates) and CI intervals (for reference traits).

FIG. 6C provides comparison of heritability estimates, utilized in accordance with various embodiments. Each point shows the reference heritability vs the heritability estimate for a particular anthropometric trait (female-only heritability in orange, male-only in light purple), horizontal error bars are reference 95% CI and vertical error bars are 2.5 to 97.5 percentile interval for the estimate. The gray line shows y=x for reference. The correlation is 0.796 between the two, and the RA2 is 0.630. Regression fit: Estimated=0.110+0.518*reference.

FIGS. 7A to 7D provides Tables 4A to 4D summarizing variant-by-sex associations identified in anthropometric traits, utilized in accordance with various embodiments. Table 4A) The number of genetic variants in females and males are listed for the four of the anthropometric traits (waist-hip-ratio, arm-fat-ratio, leg-fat-ratio, and trunk-fat-ratio). Table 4B) False-discovery rates for traits with more than five variants assigned to a component are across a range of posterior cutoffs (0.5, 0.6, 0.7, 0.8, 0.9). A posterior probability cutoff of 0.8 was used for this paper. The FDR is calculated for each of the male-specific, female-specific, and shared components separately, and indicates the false discovery rate for a variant in that component being associated with that trait. Table 4C) Model parameter estimates for pi, the proportion assigned to each component (null, female, male, and shared respectively, and the sigmasq values for the female, male, and shared components are also given. Table 4D) Overlap of genes proximal to SEMM identified variant-by-sex associations with literature interactions from Rawlik et al. (waist-hip-ratio). The table lists the numbers of genes in the reference with sex-specific associations and the number of genes overlapping with these, as well as the identities of these genes.

FIG. 8 provides selection of FDR cutoff, utilized in accordance with various embodiments. Estimated FDR at each posterior cutoff. The boxplots show the median and interquartile range at each cutoff, a gray dashed line is included to highlight an estimated FDR of 0.05.

FIGS. 9A to 9C provide validation of sex-specific variants in held-out non-British white cohort, utilized in accordance with various embodiments. Each point is the effect size estimate of a variant in the discovery (White British) vs. validation (Non-British White) cohorts, error bars show the standard errors of these estimates from GWAS summary statistics. The estimated effect sizes in females (Beta_f) and males (Beta_m) are given separately. A linear fit is shown in blue with standard error displayed in gray; the R² associated with this fit is displayed on the bottom left of each figure. Dashed white lines show the no-effect lines (0,0). Female- (FIG. 9A) and male-specific (FIG. 9B) variants in testosterone are shown separately; female-specific variants from WHR are also shown in (FIG. 9C).

FIG. 10 provides Table 5 of validation of sex-specific variants in held-out non-British white cohort, utilized in accordance with various embodiments. R² values are listed for relationship between the discovery (White British) and validation (Non-British White) effect size estimates for the set of sex-specific variants found in the discovery population (either female-specific or male-specific). No values are given for male-specific variants for the anthropometric traits because few or no male-specific variants were identified in these traits.

FIGS. 11A and 11B provide heritability and genetic correlations of biomarkers between females and males and related to menopausal status, utilized in accordance with various embodiments. FIG. 11A) SNP-based heritability estimates of 33 biomarkers for females (orange) and males (light purple). FIG. 11B) Correlation of genetic effects between males and females for 33 biomarkers.

FIG. 12 provides Table 6 of SEMM genetic parameter estimates for biomarkers, utilized in accordance with various embodiments.

FIG. 13 provides comparison of priors, utilized in accordance with various embodiments. Parameter and LOO estimates using a variety of priors (listed in the legend). The 2.5 to 97.5 percentile interval for each estimate is shown. rg is the (derived) genetic correlation. The null fraction pi[0] and repeated values (e.g. Sigma[2,1]) are not included. ELPD-DIFF is the difference in the estimated log predictive density calculated with leave one out cross-validation relative to the best model, and a 95% CI for the difference is included.

FIG. 14 provides genetic correlations of biomarkers, utilized in accordance with various embodiments. The genetic correlation within women (pre- vs. post-menopausal, in pink) was higher or equal to than either that between both post-menopausal women and men (green) and pre-menopausal women and men (tan). Error bars in all three plots indicate the 2.5 to 97.5 percentile interval from STAN sampling.

FIG. 15 provides Table 7 of genetic correlations including menopausal status, utilized in accordance with various embodiments. The data was divided into pre-menopausal women, post-menopausal women, and men, and SEMM was fit to these data to calculate genetic correlations between pre- and post-menopausal women (rg.pre_post), pre-menopausal women and men (rg.pre_male), and post-menopausal women and men (rg.post_male). The 2.5 to 97.5 percentile intervals are given for each value with/suffix indicating the lower limit of the interval and u suffix indicating the upper limit. The proportion assigned to the non-null component (pi[1]) is also provided.

FIG. 16 provides Table 8 of summary of variant-by-sex-effect associations in biomarker traits, utilized in accordance with various embodiments.

FIG. 17 provides estimated effect sizes for genetic variants with non-zero effects on testosterone, utilized in accordance with various embodiments (x-axis, estimated effect size in females; y-axis, estimated effect size in males). Light purple dots correspond to variants that belong to the “Male-specific” effect component; orange corresponds to the “Female-specific” effect component; and gray dots correspond to genetic variants that belong to the “Shared” effect component of SEMM.

FIG. 18 provides a Miami plot showing sex-specific variants associated with testosterone levels, utilized in accordance with various embodiments. P-values for females are shown on the top half of the plot, and males are shown on the bottom half of the plot with inverted p-values. Light purple dots are male sex-specific variants, orange dots are female-specific variants, and magenta dots are those assigned to the shared component. Light gray dots are variants that were excluded from the model fitting process because they are in LD with other variants. Each variant is displayed in both the male and female portions of the plot; we can use this to visualize the difference in p-values for the effects. P-values <10⁻³⁰ were truncated to 10⁻³⁰ for easier visualization; and the names of genes proximal to variants with p<10⁻²° in either males or females are shown.

FIG. 19 provides results of Mendelian Randomization tests with sex-specific testosterone variants as instruments on all analyzed outcomes, utilized in accordance with various embodiments. This plot shows the Mendelian Randomization results for all traits, each trait is shown separately. Effect sizes (betas) are estimated using either the female or male-specific variants as instrumental variables for testosterone exposure. 95% confidence intervals are shown for each estimate. Points are colored by the sex of the outcome population (light purple for males, orange for females, and gray for combined), with size indicating the −log 10 p-value, and shape showing the source of the GWAS statistics for the trait (triangles for UKBB=UK Biobank, circles for all others). Trait/exposure pairs that are significant after multiple hypothesis correction are indicated with an asterisk.

FIG. 20 provides Mendelian Randomization results where sex-specific testosterone variants show evidence of a causal effect on the trait, utilized in accordance with various embodiments. Each plot shows the SNP effects for one set of variants (either male or female variants) and one outcome or trait. Mendelian Randomization fits are shown for Inverse variance weighted (light blue), inverse variance weighted fixed effects (blue), and MR Egger (light green). Female-specific variants show evidence of a causal association with height, waist circumference, hip circumference, and body mass index (BMI); male-specific variants show evidence of causal associations with height and Type 2 Diabetes (DIAGRAMplusMetaboChip). For height and BMI, both outcomes from the UK Biobank and GIANT consortia show associations, and for BMI GIANT data both male and combined populations show this relationship.

FIG. 21 provides comparison of sex-specific and combined polygenic risk scores in accordance with various embodiments. The sex-specific PRS (x-axis) and the combined PRS (y-axis) are shown for the individuals in the test set for males and females. The two PRSs are centered and scaled so each has zero mean and unit variance. The red diagonal line indicates y=x.

FIGS. 22A and 22B provide polygenic risk score (PRS) predictions for testosterone in male (FIG. 22A) and female (FIG. 22B) individuals for male-specific (light purple), female-specific (orange), and combined (gray) PRS models in accordance with various embodiments. Plots show predicted stratified risk bins for testosterone levels (x-axis) versus mean covariate-adjusted testosterone for those individuals (y-axis). These values were calculated on a held-out test set of unrelated White British individuals. The error bars represent standard errors.

FIG. 23 provides sex-specific polygenic prediction with simplified covariates in accordance with various embodiments. The mean testosterone values are shown (y-axis) across stratified risk bins (x-axis) based on sex-specific PRS (light purple for male PRS and orange for female PRS) or the combined PRS (gray) for male and female individuals separately. Mean values and PRS scores are calculated on a held-out test data set of unrelated White British individuals and the testosterone are shown as log₁₀-transformed values of residuals after the covariate adjustment using a reduced set of covariates (age, genotype PCs 1-10). The error bars represent standard errors. The PRSs are computed with 8236, 7169, and 7320 genetic variants for male-specific, female-specific, and the combined model, respectively.

FIG. 24 provides comparison of priors, utilized in accordance with various embodiments. Parameter and LOO estimates using a variety of priors (listed in the legend). The 2.5 to 97.5 percentile interval for each estimate is shown. rg is the (derived) genetic correlation. The null fraction pi[0] and repeated values (e.g. Sigma[2,1]) are not included. ELPD-DIFF is the difference in the estimated log predictive density calculated with leave one out cross-validation relative to the best model, and a 95% CI for the difference is included.

FIG. 25 provides tissue specific enrichment analysis (TSEA) for sex-specific genetic effects on testosterone levels, utilized in accordance with various embodiments. Visualization of tissue-specific enrichment for male-specific genetic effects on testosterone levels created by the TSEA tool. Each node is a tissue, the color of the tissue colors the enrichment of the set of genes within the genes that are specific to that tissue (as indicated by a Benjamini-Hochberg corrected Fisher's exact test p-value). The size of the node corresponds to the number of enriched transcripts, each node contains three subsections, these represent three different stringency thresholds for the selection of tissue-specific genes. The topography of the diagram is generated from hierarchical clustering of the transcripts in each tissue, nodes closer to each other share more transcripts. Liver is highlighted with significant enrichment in males (p=3.91×10⁻⁷) and but not females (p>0.1).

### DETAILED DESCRIPTION

Turning now to the drawings and data, methods and processes to determine hormone levels from genetic data and performing clinical assessments and/or treatments based on hormone levels are provided. In many embodiments, nucleic acids (e.g., DNA or RNA) are sequenced and variants are identified, which are utilized in a linear model to determine hormone levels. Clinical assessments and treatments can be performed based on hormone level.

Various embodiments are directed towards identification of therapeutic targets based on hormone physiology. In some embodiments, various genes are targeted that have been discovered to alter (i.e., increase or decrease) hormone levels in an individual. Accordingly, a number of embodiments are directed towards agonists and/or antagonists of gene products to achieve a desired alteration in hormone level.

### Genetic Indication of Hormone Level

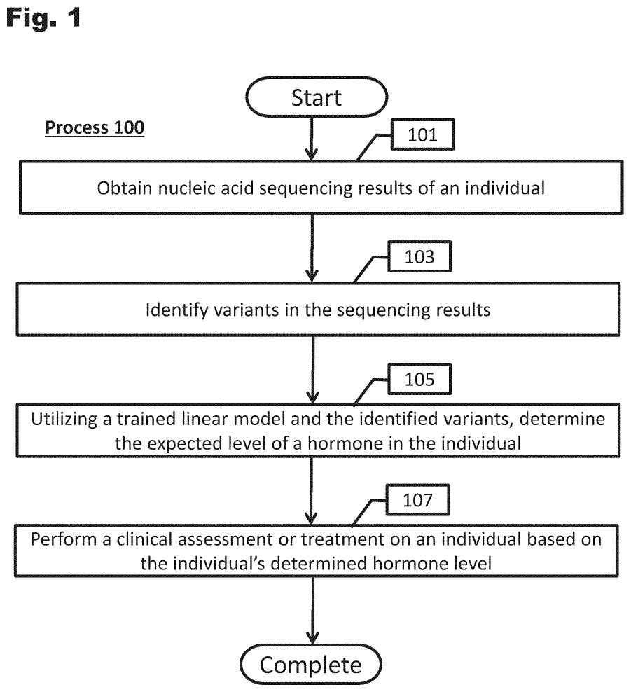

Several embodiments are directed towards determining an individual's hormone level based on their genetic sequence. It is now known that a number of variants within a genetic sequence can indicate an alteration of hormone level as compared to the population average or median. By sequencing an individual's nucleic acids, variants can be identified and used in a trained computational model to determine the hormone level of the individual. A number of hormones can be analyzed, including (but not limited to) testosterone, estrogen, oxytocin, vasopressin, and progesterone. One exemplary method is depicted in FIG. 1.

As shown in FIG. 1, Process 100 obtains 101 an individual's genetic sequence data. The data, in accordance with many embodiments, is any nucleic acid sequence data of individual. In some embodiments, genetic data is an individual's whole genome, a partial genome, exome, or other data that is inclusive of variant data. In some embodiments, genetic data is only sequencing data on a set of loci that have been found to be important to determine the level of the hormone(s) to be analyzed (e.g., capture sequencing). In some embodiments, sequence data are obtained by a biopsy of an individual, in which genetic material is extracted from the biopsy and sequenced.

In accordance with various embodiments, genetic data can be derived from a number of sources. In some instances, these genetic data are obtained de novo by extracting the DNA or RNA from a biological source and sequencing it. Alternatively, genetic sequence data can be obtained from publicly or privately available databases. Many databases exist that store datasets of sequences from which a user can extract the data to perform experiments upon, such as the U.K. Biobank. In many embodiments, the genetic sequence data include whole or partial genomes that include variants to be examined; accordingly, any genetic data set as appropriate to the requirements of a given application could be used.

In accordance with various embodiments, an individual's genetic sequence data are processed 103 to identify variants. In many embodiments, an individual's variant profile is further analyzed and trimmed, often dependent on the application. In some embodiments, variant calls within repeat regions are removed. In some embodiments, indels are removed. In some embodiments, only variants of a particular frequency (e.g., rare variants with MAF 1.0%) are examined and thus all other variants are excluded. In some embodiments, known and/or pre-classified variants from known various databases are removed. For example, it is now known that various variants affect males but not females and vice versa, thus when examining variants related to a particular hormone analysis, it may be ideal to focus on variants that are specific to sex of the individual being analyzed.

The expected hormone level in the individual is determined 105 utilizing a trained linear model and the identified variants. In some embodiments, a trained linear model is any classification model capable of determining hormone levels based on genetic variants. In a number of embodiments, an individual's genetic sequence data are entered into a computational model, wherein subsequently the expected hormone level of the individual's variants are determined.

In a number of embodiments, a trained linear model provides a polygenic risk score that provides an indication of the level of hormone level of the individual. In some embodiments, batch screening iterative lasso (BASIL) is used to train the linear model. In some embodiments, the linear model is a multivariate penalized regression, but it should be understood that any appropriate predictive model can be utilized.

In several embodiments, a linear model is trained utilizing genetic variant data derived from a collection of individuals in which their hormone level has been recorded. It has been found that prediction of hormone level from genetic variants is better in some cases when a sex-specific model is utilized. That is, in some instances, models that are trained utilizing variants derived from individuals of only one sex (i.e., male or female) provide better prediction of hormone level than models trained on both sexes. As detailed within the Exemplary Embodiments section herein, various variants are robustly predictive of hormone level for one sex but less predictive for the other. Accordingly, in some embodiments, a linear model is trained specifically for one sex (i.e., male or female) based on the variants identified in a collection of individuals of that sex. It should be understood, however, that various embodiments also utilize a linear model that has been trained utilizing genetic variant data derived from individuals of both sexes.

Based on the individual's determined hormone level, a clinical assessment or a treatment is performed 107 on the individual. Clinical assessments to be performed include (but are not limited to) blood tests, medical imaging, and clinical tests to detect diabetes (especially type II), insulin resistance, dyslipidemia, hypertension, polycystic ovarian syndrome (PCOS) disease, virilization, amenorrhea, hirsutism, hypothyroidism, hypopituitarism, delayed puberty, precocious puberty, premenstrual dysphoric disorder (PMDD), erectile dysfunction, female sexual dysfunction, hypogonadism, and infertility. Treatments that can be performed include increasing or decreasing a hormone level utilizing a medication or dietary supplement. Treatments to increase testosterone include (but not limited to) testosterone replacement therapy (TRT), methyltestosterone, finasteride, mesterolone, cyproterone, vitamin D, and caffeine. Treatments to decrease testosterone include glucocorticosteroids, metformin, oral contraceptives, and spironolactone.

While specific examples of determining an individual's hormone level are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for determining an individual's hormone level appropriate to the requirements of a given application can be utilized in accordance with various embodiments of the invention.

Pathological Targets for Altering Hormone Levels

Various embodiments are directed towards treatments that are directed towards targets involved in the pathophysiology of hormone levels. Based on recent findings as detailed in the Exemplary Embodiments described herein, it is now understood that several gene products involved in hormone regulation can be targeted. Furthermore, in many instances, gene products to be targeted are sex specific. In other words, altering the activity (i.e., increasing or decreasing) of several gene targets would have a much greater effect in one sex and less effect in the other sex.

It is now understood that agonizing or antagonizing expression or activity of certain genes can be performed to lower testosterone in a female. For instance, to lower testosterone in a female, the expression or the activity of the following genes (or products thereof) can be agonized: LIPE, FBXO15, and DCAF12. To lower testosterone in a female, the expression or the activity of the following genes (or products thereof) can be antagonized: TRMU, SLC22A11, ZAN, STAG3, CYP3A43, POR, MCM9, MFSD9, and PER3. To lower testosterone in a male, the expression or the activity of the following genes (or products thereof) can be antagonized: ANXA9, BCO2, EML4, GAB1, HNF4A, JMJD1C, MACF1, METTL21A, NF1, P2RX7, PP1P5K1, QARS, RPTN, and SLCO1A2. To increase testosterone in a male, the expression or the activity of the following genes (or products thereof) can be antagonized: ACSL5, DNAH2, HGFAC, KIF27, KLHL38, LRRC14, MARS, MSH5, PPP2R3A, PRMT6, SERPINA1, SERPINF2, SLC17A2, SLCO1A2, SOS2, SPAST, SPDL1, TGM7, USP8, YY1AP1, ZBTB10, and ZCCHC6.

### Applications and Treatments Related to Aging Processes

Various embodiments are directed to performing a clinical assessment and/or treatment based on determining hormone levels of an individual from genetic data. As described herein, an individual's hormone level is determined by a linear model. Based on one's hormone level, an individual can be subjected to further diagnostic testing and/or treated with various medications, dietary supplements, an exercise regimen, a lifestyle change and any combination thereof.

### Clinical Diagnostics, Medications and Supplements

Several embodiments are directed to the use of medications and/or dietary supplements to treat an individual based on their hormone level. In some embodiments, medications and/or dietary supplements are administered in a therapeutically effective amount as part of a course of treatment. As used in this context, to “treat” means to ameliorate at least one symptom of the disorder to be treated or to provide a beneficial physiological effect. For example, one such amelioration of a symptom could increase or decrease the level of a hormone.

A therapeutically effective amount can be an amount sufficient to prevent reduce, ameliorate or eliminate the symptoms of diseases or pathological conditions susceptible to such treatment, such as, for example, correcting hormone levels. In some embodiments, a therapeutically effective amount is an amount sufficient to correct hormone, as would be determined by the measurements of the hormone over time.

A number of medications are available to treat an individual with a low or high hormone level. In some embodiments, treatments to increase testosterone include (but not limited to) testosterone replacement therapy (TRT), vitamin D, and caffeine. In some embodiments, treatments to decrease testosterone include glucocorticosteroids, metformin, oral contraceptives, and spironolactone.

Numerous clinical and laboratory assessments may also be performed, which can determine whether an individual has a particular disorder. Clinical assessments to be performed include (but are not limited to) blood tests, medical imaging, and clinical tests to detect insulin resistance, dyslipidemia, hypertension, polycystic ovarian syndrome (PCOS) disease, virilization, amenorrhea, hirsutism, hypothyroidism, hypopituitarism, delayed puberty, precocious puberty, premenstrual dysphoric disorder (PMDD), erectile dysfunction, female sexual dysfunction, hypogonadism, and infertility.

Numerous lifestyle regimens may also help to ameliorate or treat low or high hormone levels for an individual. Those include but not limited to changing exercise level, changing the type and/or duration of exercise, and changing body weight and fat composition.

### EXEMPLARY EMBODIMENTS

The various embodiments will be better understood with the examples provided within. Many exemplary results of computational models to predict hormone levels from genetic data are described.

### Example 1: Sex-Specific Genetic Effects Across Biomarkers

Sex differences have been shown in laboratory biomarkers; however, the extent to which this is due to genetics is unknown. In this example, sex-specific genetic parameters (heritability and genetic correlation) were inferred across 33 quantitative biomarker traits in 181,064 females and 156,135 males from the UK Biobank study. A Bayesian mixture model, Sex Effects Mixture Model, was applied to Genome-wide Association Study summary statistics in order to (1) estimate the contributions of sex to the genetic variance of these biomarkers and (2) identify variants whose statistical association with these traits is sex-specific. It was found that the genetics of most biomarker traits are shared between males and females, with the notable exception of testosterone, where 119 female and 445 male-specific variants were identified. These include protein-altering variants in steroid hormone production genes (POR, UGT2B7). Using the sex-specific variants as genetic instruments for Mendelian Randomization, evidence for causal links between testosterone levels and height, body mass index, waist and hip circumference, and type 2 diabetes were found. It is also shown that sex-specific polygenic risk score models for testosterone outperform a combined model. Overall, these results demonstrate that while sex has a limited role in the genetics of most biomarker traits, sex plays an important role in testosterone genetics.

### Introduction

Sex differences have been documented in many phenotypes and diseases. Across quantitative traits, men and women typically have overlapping distributions with different means, examples of these traits include height and body mass index (BMI). Previous studies have demonstrated that some of this difference is due to sex-specific genetic factors. Genome-wide Association Studies (GWAS) are increasingly used to identify variants that contribute to sex differences, and recently, gene-by-sex interactions have been identified across many phenotypes, including in anthropometric traits, irritable bowel syndrome, and glioma.

Examination of blood and urine laboratory biomarker levels reveal sex differences (N. Sinnott-Armstrong, et al., bioRxiv 660506 (2019), the disclosure of which is incorporated herein by reference); however, it is unknown to what extent these sex differences are related to underlying differences in the genetic architecture versus environmental differences. Heritability, or the fraction of phenotypic variability explained by genetic variance, was initially estimated from family studies; but now, with the increasing availability of genome-wide data, common genetic variants (such as single nucleotide polymorphisms (SNP)) are used for this estimation. Methods for estimating SNP-based heritability include LD-score regression, GREML (genomic related matrix restricted maximum likelihood), Haseman-Elston Regression, and the moment-matching approach (B. Bulik-Sullivan, bioRxiv 018283 (2015); W. G. Hill, Genetical Research 32, 265-274 (1978); G. Ni, et al., Am. J. Hum. Genet. 102, 1185-1194 (2018); and D. speed and D. J. Balding, Nat. Genet. 51, 277-284 (2019); the disclosures of which are incorporated herein by reference). These methods are applied to a sample of unrelated individuals in order to quantify the proportion of phenotypic variance explained by all genetic variants in the GWAS.

At a trait-level, sex-specific heritability and between-sex genetic correlation can be used to examine what fraction of the genetics of that trait is shared. The UK Biobank is a prospective population-based study of 500,000 individuals that includes both genetic and phenotypic data, allowing for rich SNP-based estimation of heritability. While most traits do not show sex effects on heritability, previous studies have documented these differences in a subset of traits, including fat distribution and other anthropometric traits. However, there has yet to be an analysis of these sex differences across biomarkers.

Herein is described an approach for estimating the extent to which genetic effects are correlated between sexes and identifying the proportion of relevant variants that have shared effects versus effects that are specific to each sex. This approach was applied to blood and urine biomarker data from the UK Biobank to examine sex differences in genetic effects, and find differences primarily in the genetic determinants of testosterone level. Furthermore, these identified sex differences were used to provide hypotheses about biological mechanisms including (1) examination of protein-altering variants and tissues where these genes are selectively expressed, (2) causal inference using Mendelian Randomization to assess relationships between testosterone and other traits, and (3) improved genetic risk prediction models for testosterone.

Materials and Methods

Genotype data. Genotype data from the UK Biobank dataset release version 2 and the hg19 human genome reference was used for all analyses in the study (K. Rawlik, O. Canela-Xandri, and A. Tenesa, Genome Biol. 17, 166 (2016), the disclosure of which is incorporated herein by reference). To minimize the variability due to population structure in the dataset, the analyses was restricted to unrelated White British individuals (as indicated by self-reported ethnicity, UKBB field ID 21000) without missing data. Variant annotations, filtering, and LD pruning were performed as previously described (C. DeBoever, et al., Nat. Commun. 9, 1612 (2018); and Y. Tanigawa, et al., bioRxiv 442715 (2019); the disclosures of which are incorporated herein by reference). An additional filter for variants with Hardy-Weinberg Equilibrium <10⁻⁷ and less than 1% missingness was utilized; and plink -xchr-model 2 was used.

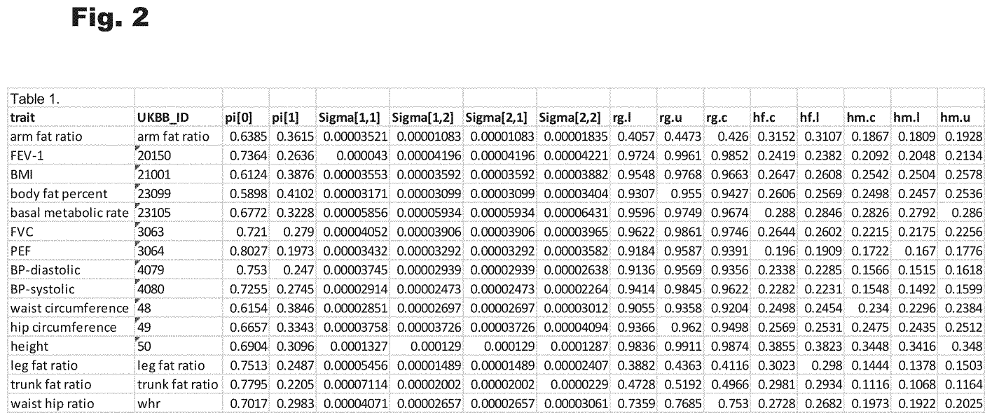

Anthropometric traits. To demonstrate the utility of the method, SEMM was applied to previously examined anthropometric traits (M. Rask-Andersen, et al., Nat. Commun. 10, 339 (2019), the disclosure which is incorporated herein by reference; and K. Rawlik, O. Canela-Xandri, and A. Tenesa (2016), cited supra). (field IDs in Table 1 of FIG. 2).

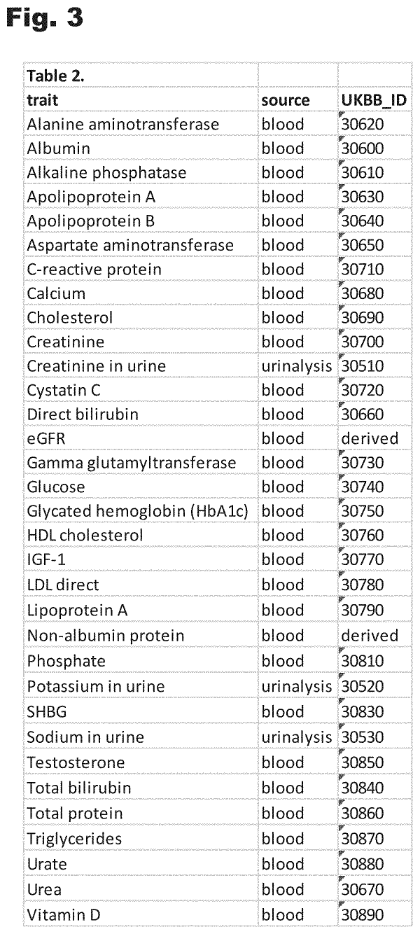

Selection and Processing of Biomarker Traits. The study focused on 33 of 38 biomarkers previously described and covariate-adjusted (N. Sinnott-Armstrong, et. al., (2019), cited supra) (field IDs in Table 2 of FIG. 3).

Menopause phenotype definition. A stringent definition was used to dividing individuals into pre- vs post-menopause menopause to separate into clear categories and avoid including peri-menopause.

Summary statistic generation. Genome-wide association summary statistics were generated separately for males and females using PLINK v2.00aLM (2 Apr. 2019). Age, genotyping array used, and the first four PCs were included as covariates. Variants with missing standard errors or standard errors >0.2 in either sex were also removed.

Sex Effects Mixture Models (SEMM). A two-component mixture model consisting of a point mass centered at zero and a multivariate normal distribution was used to estimate the variance-covariance matrix, from which the genetic correlation and heritability was estimated. This model was extended to a four-component model with male- and female-specific components in order to identify variants with sex-specific genetic effects.

Sex-specific multivariate polygenic prediction. To construct sex-stratified polygenic risk score (PRS) models using multivariate penalized regression, a random split dataset was created of White British individuals in UK Biobank into 70% training, 10% validation, and 20% test sets. Covariate adjustment testosterone residual values was used as described previously (N. Sinnott-Armstrong, et. al., (2019), cited supra); however, adjust was performed for both sex-separated and combined cohorts.

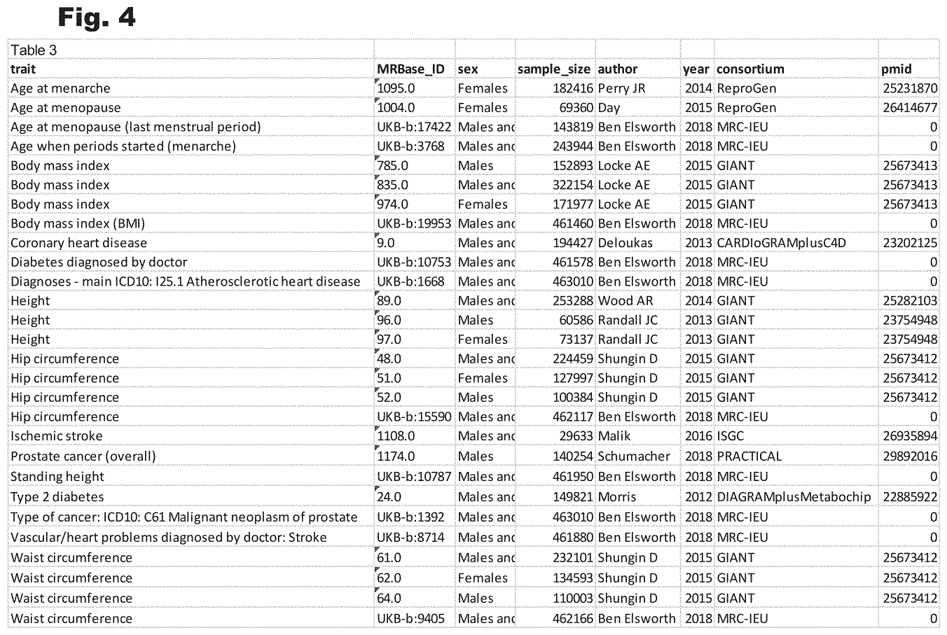

Mendelian Randomization. MR-Base was used to test for evidence for causal associations between testosterone and 10 outcomes of interest using the sets of female- and male-specific testosterone variants (G. Hemani, et al., Elife 7, e34408 (2018), the disclosure of which is incorporated herein by reference). Variants were pruned for LD with clumping and the analysis was performed with MR Egger, Inverse Variance Weighted, and Inverse Variance Weighted with fixed effects. For each of the outcomes, summary statistics were used from both a UK Biobank and non-UK Biobank source and sex-divided outcomes where available. The traits include: waist circumference, hip circumference, height, body mass index, age at menarche, age at menopause, prostate cancer, heart disease, type 2 diabetes, and stroke (see D. Shungin, et al., Nature 518, 187-196 (2015); A. Wood, et al., Nat. Genet. 46, 1173-1186 (2014); J. C. Randall, et al., PLoS Genet. 9, e1003500 (2013); A. E. Locke, et al., Nature 518, 197-206 (2015); J. Perry, et al., Nature 514, 92-97 (2014); F. R. Schumacher, et al., Nat. Genet. 50, 928-936 (2018); CARDIoGRAMplusC4D Consortium, et al., Nat. Genet. 45, 25-33 (2013); A. Morris, et al., Nat. Genet. 44, 981-990 (2012); and R. Malik, et al., Neurology 86, 1217-1226 (2016); the disclosures of which are incorporated herein by reference) (Table 3 in FIG. 4). A Bonferroni correction was used to account for multiple tests (p-value <0.05/168=2.98×10⁻⁴).

### Results

### Sex Effect Mixture Models

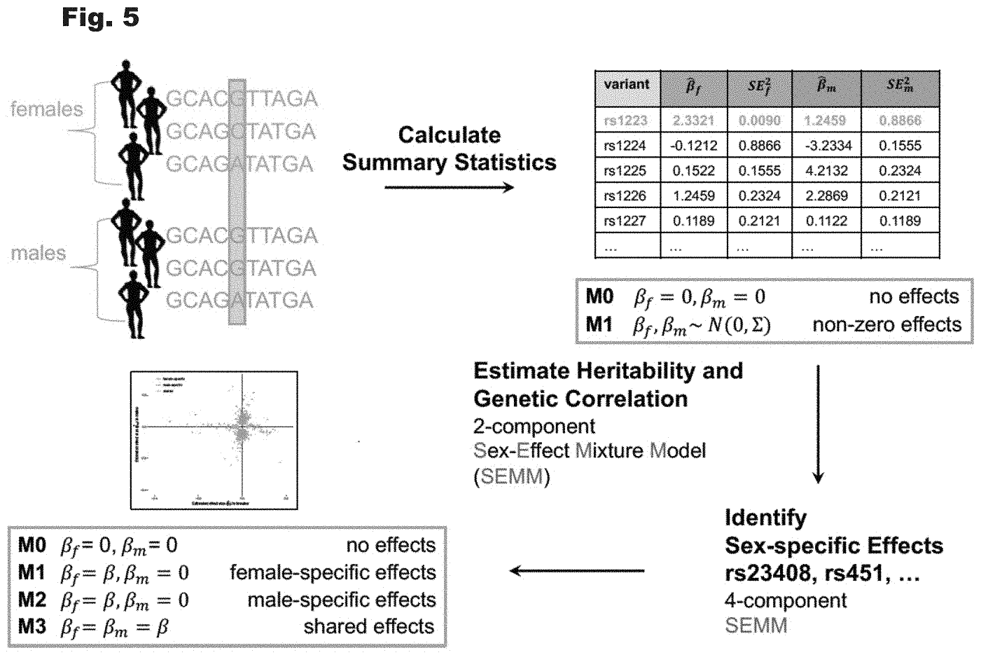

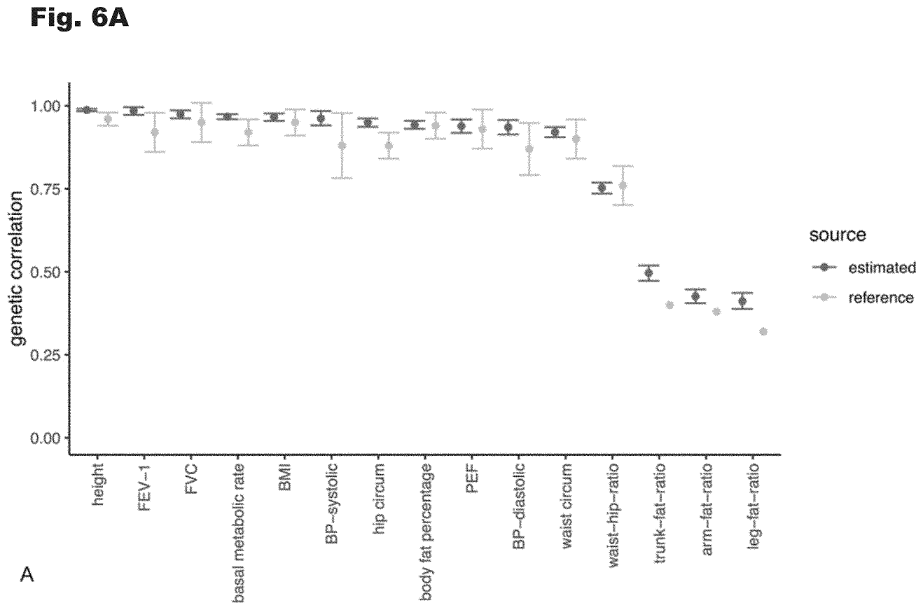

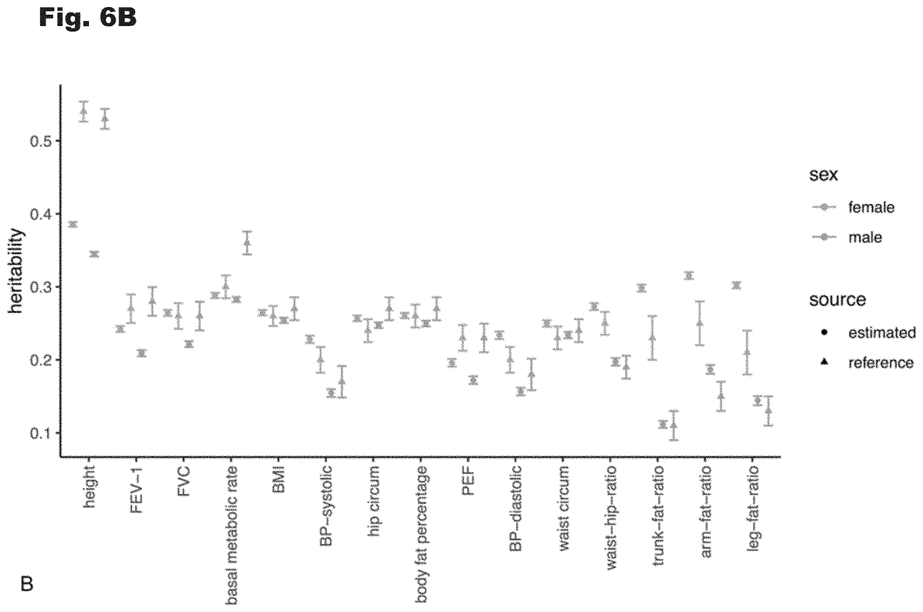

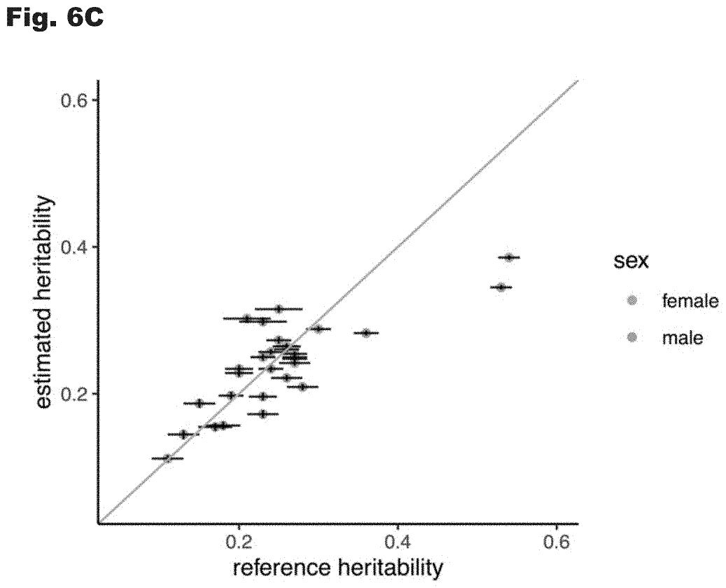

A two-component Bayesian Sex Effect Mixture Model (SEMM) was built to estimate the contributions of sex to genetic variance using GWAS summary statistics (FIG. 5; SEQ. ID NOS 1-6). The model contains a null component, for variants that do not contribute to the trait, and a non-null component, for variants that represent the genetic contribution to that trait. Variants driving male and female traits in the non-null component are modeled as two-dimensional vectors drawn from a multivariate normal distribution with a variance-covariance matrix that can be used to estimate the genetic correlation between sexes. To assess whether the approach obtains reliable estimates in real data, SEMM was applied to traits from Rawlik et al. (2016, cited supra) and obtained overlapping genetic correlations and similar but not identical heritability estimates (Table 1 in FIG. 2; FIGS. 6A-6C).

The two-component SEMM was extended to a four-component model to identify genetic variants with different effects in males and females (FIG. 5). To do so, two components for detecting genetic variants that have stronger effects in one sex were added. Similar to the two-component model, the four-component SEMM also contains a no-effect and shared-effect component. Through fitting this model, genetic variants that have “shared” effects, where the variant or set of variants have the same effect in males and females, and “sex-specific” effects, where the variants have different effects in males than in females were able to separated (e.g. this variant is associated with higher lab values in females but not in males).

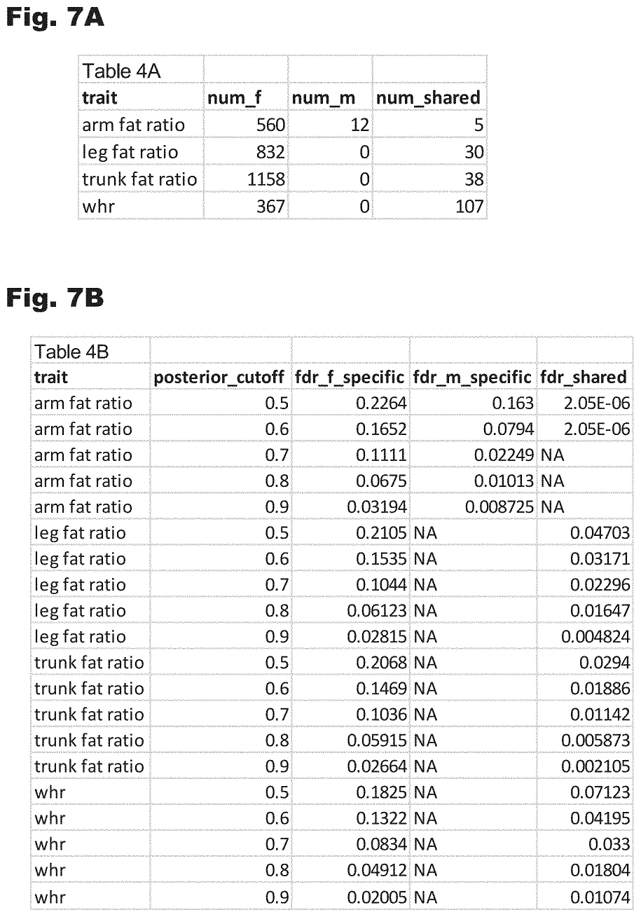

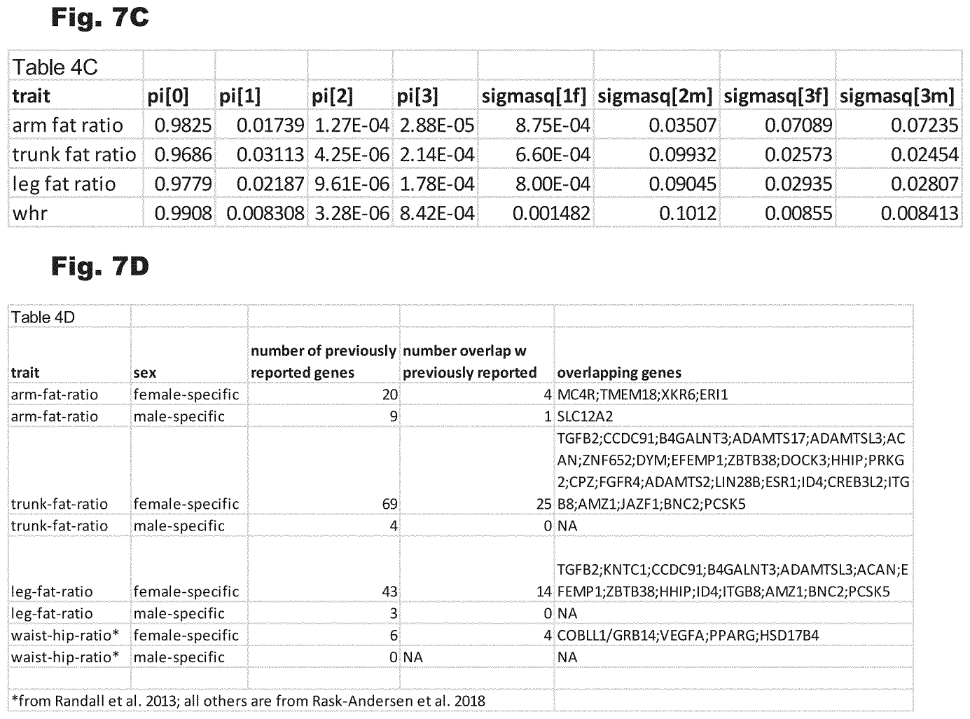

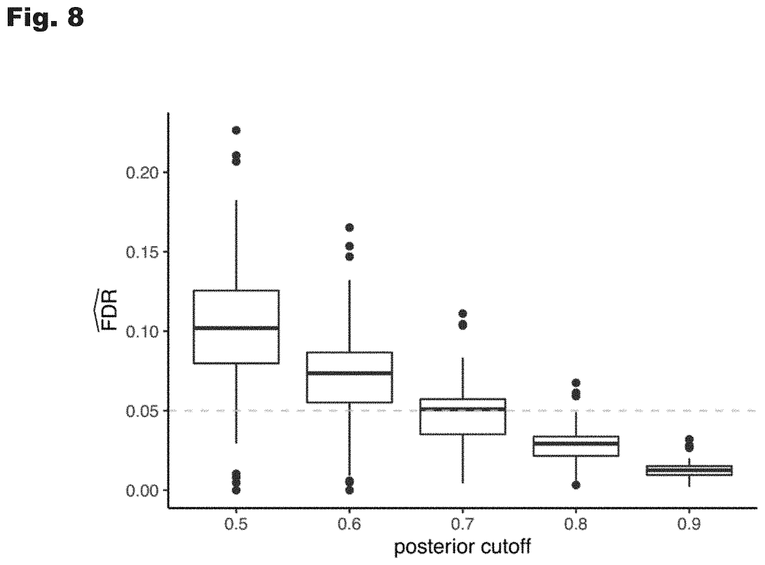

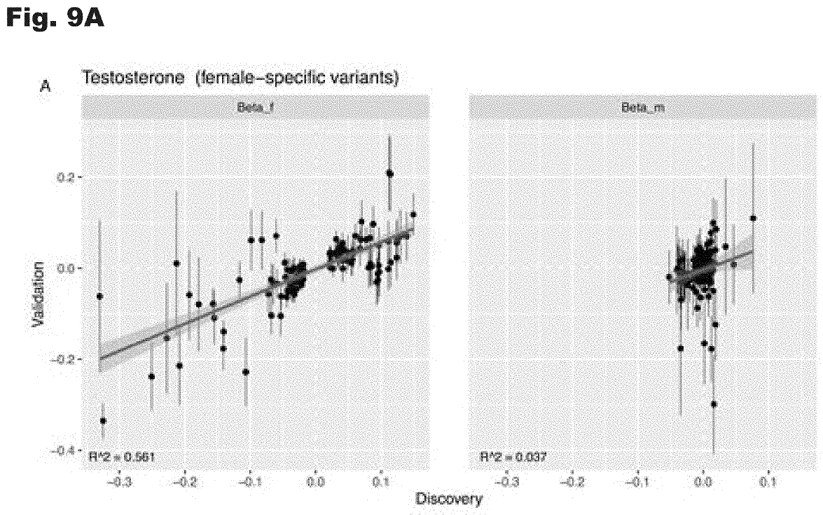

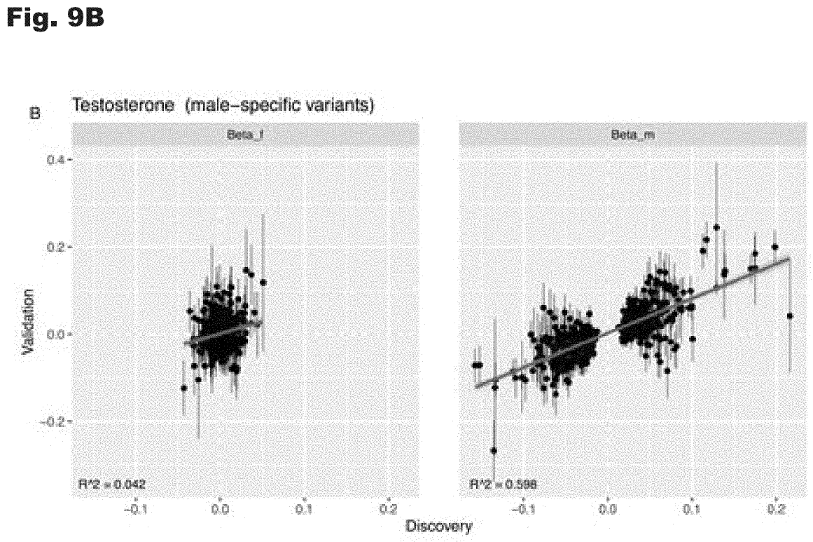

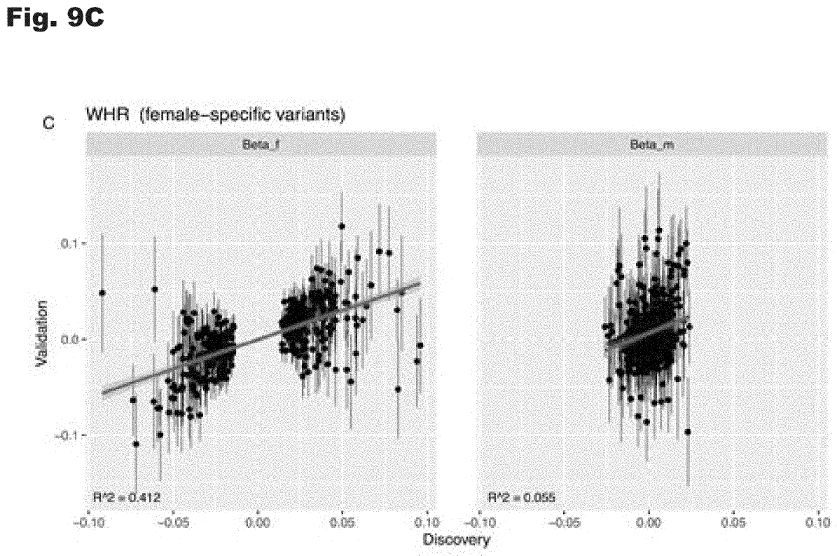

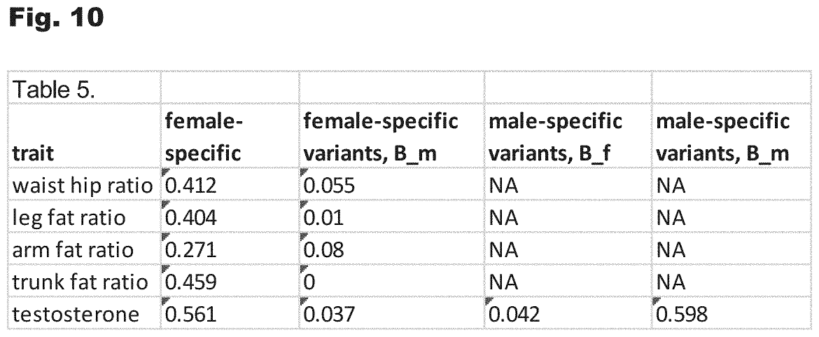

To demonstrate the efficacy of the four-component SEMM, the model was applied to four traits, waist-hip ratio, arm-fat-ratio, leg-fat-ratio, and trunk-fat-ratio with previously identified sex-specific genetic effects. 367, 560, 832, and 1,158 genetic variants were identified that had significantly stronger associations in females in waist-hip-ratio, arm-fat-ratio, leg-fat-ratio, and trunk-fat-ratio, respectively. In males, only 12 variants were found in arm-fat-ratio (estimated False Discovery Rate 4.9-6.8% across all traits, see Tables 4A-4D of FIGS. 7A-7D, and FIG. 8). Included in the female-specific waist-hip ratio variants were genetic variants proximal to four of six previously reported genes (COBLL1/GRB14, VEGFA, PPARG, HSD17B4). Fat ratio variants were proximal to one of the male- and 48 of the female-specific genes previously identified, indicating that we capture known sex-specific signal (see Table 4D of FIG. 7D for the overlap). Additionally, these sex-specific variants were validated by showing they have similar effect sizes in a held-out cohort (see FIGS. 9A-9C, Table 5 of FIG. 10).

### Sex-Differential Heritability

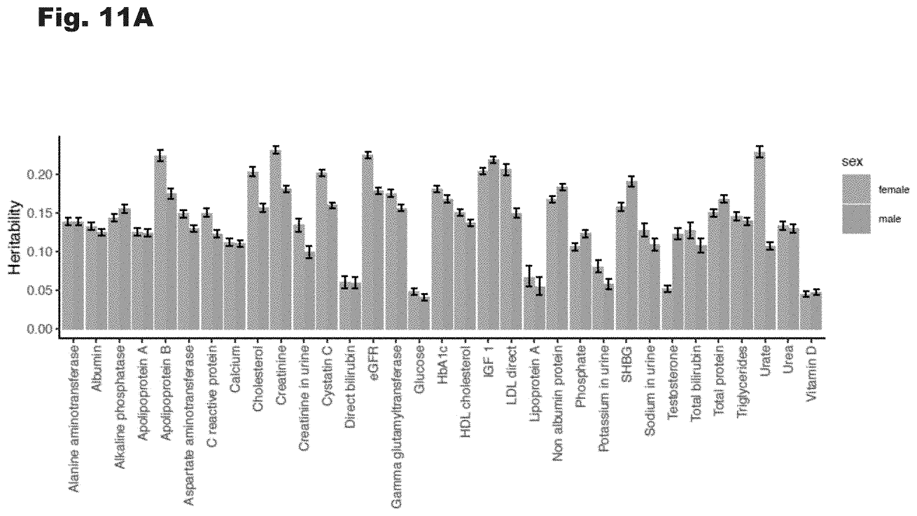

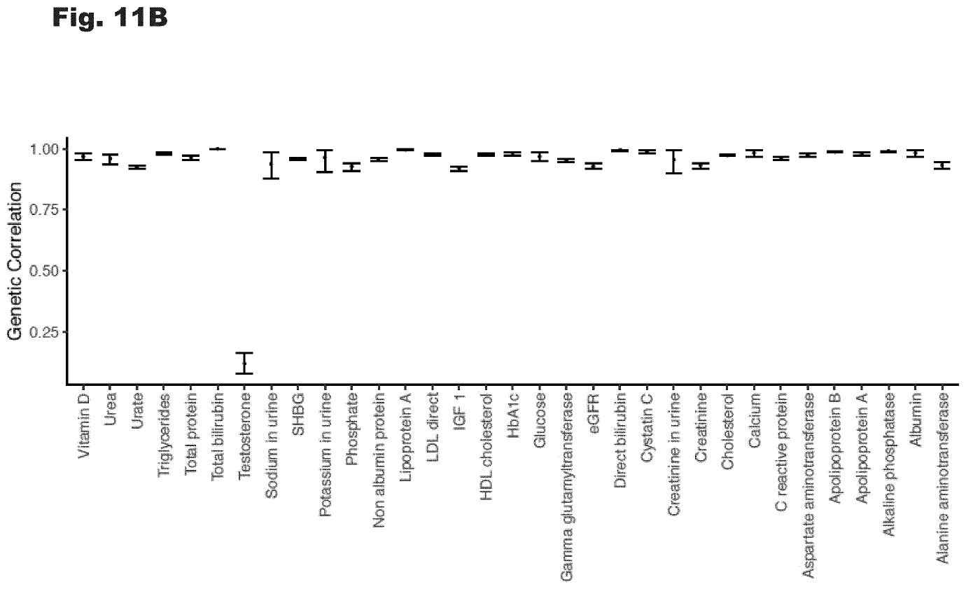

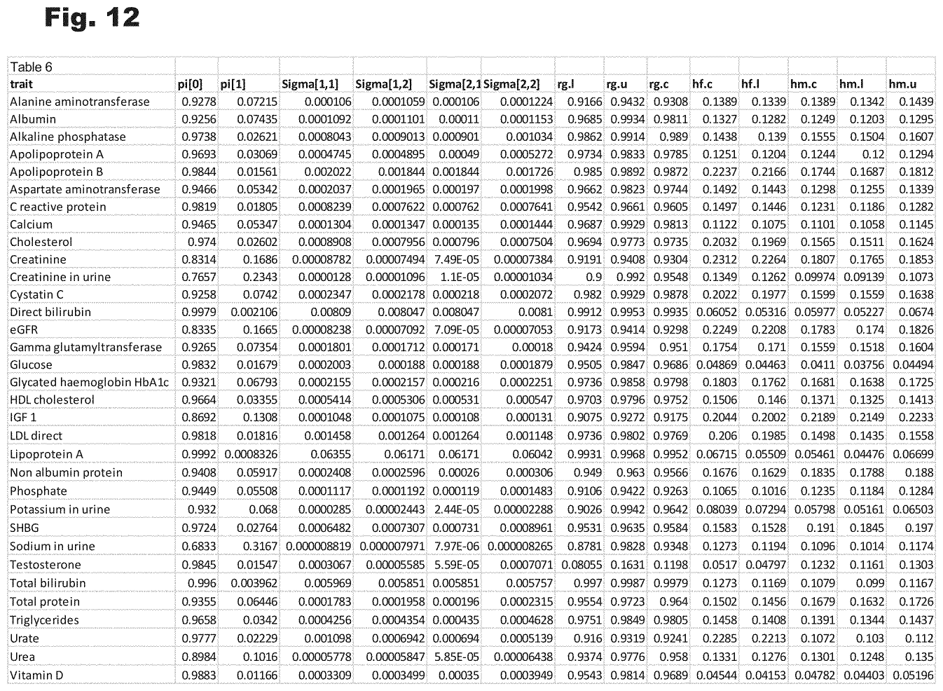

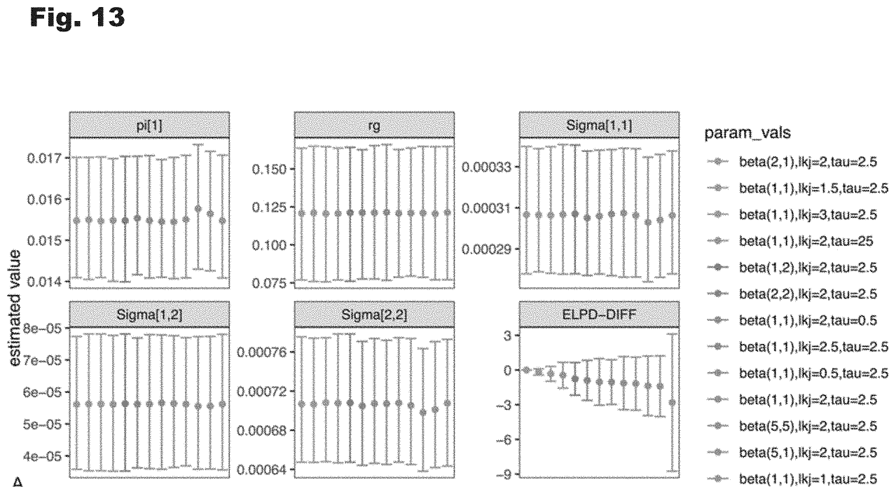

The two-component SEMM was applied to 33 UK Biobank biomarkers in order to estimate the sex specific heritability and genetic correlation for each trait (see Table 2 of FIG. 3 for a full list of these traits). While a large fraction of biomarkers had overlapping heritability estimates, sex differences were found in the heritability of 17 of 33 biomarkers, including testosterone, IGF-1, non-albumin protein, SHBG, total protein (higher in males), apoplipoprotein B, C-reactive protein, cholesterol, creatinine, cystatin C, eGFR, gamma glutamyltransferase, HDL-C, LDL-C, potassium in urine, sodium in urine, urate (higher in females) (FIG. 11A). Of these, cholesterol, creatinine and sodium in urine, LDL, testosterone, and urate showed greater than 1.3-fold differences. For the majority of traits, the between-sex genetic correlations were close to 1.0, indicating shared additive genetic effects between males and females (FIG. 11B). By contrast, for testosterone, a genetic correlation of only 0.120 was estimated (2.5 to 97.5 percentile interval: 0.0805 to 0.163), indicating largely non-overlapping genetic effects between males and females (see Table 6 of FIG. 12; these estimates are consistent across priors: FIG. 13).

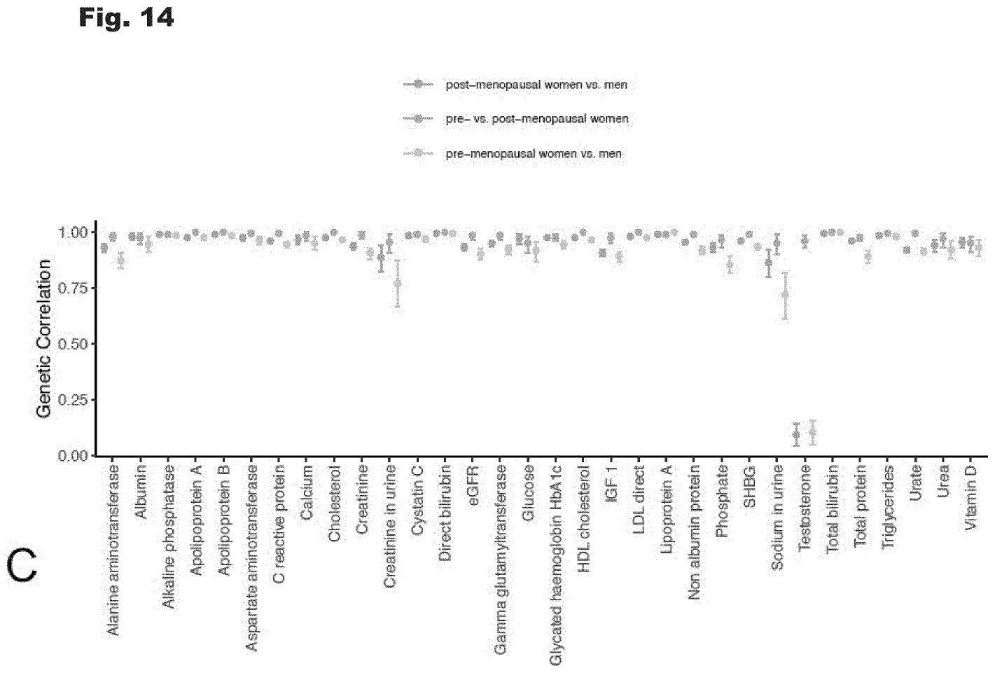

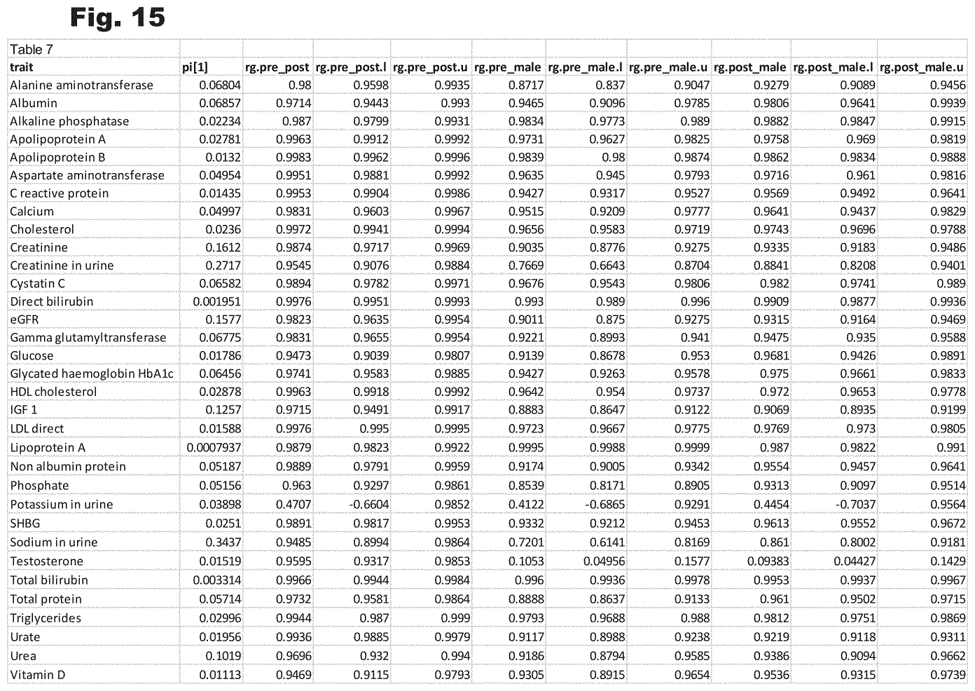

The heritability of a particular trait can vary across the lifetime, as genetics may explain more or less of the variation in that particular trait. Previous studies have found that pre- and post-menopausal women have different heritability for BMI, waist and hip measures and lipid biomarkers (L. E. Kelemen, et al., BMC Medical Genetics 11, 156 (2010), the disclosure of which is incorporated herein by reference). To examine this across biomarkers in the UK Biobank population, we applied our two-component SEMM to summary statistics for pre- and post-menopausal women. We found that genetic correlations between pre- and post-menopausal women close to 1.0, and all traits had higher or equivalent within-sex (between pre- and post-menopausal women) than between-sex (between either group and men) genetic correlations (FIG. 14, Table 7 of FIG. 15).

Identification of Genetic Variants with Sex-Specific Effects

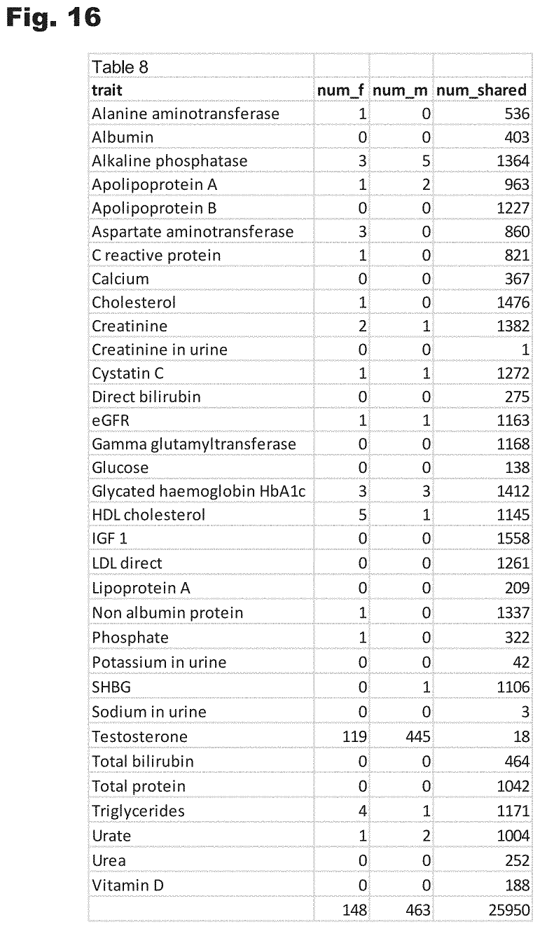

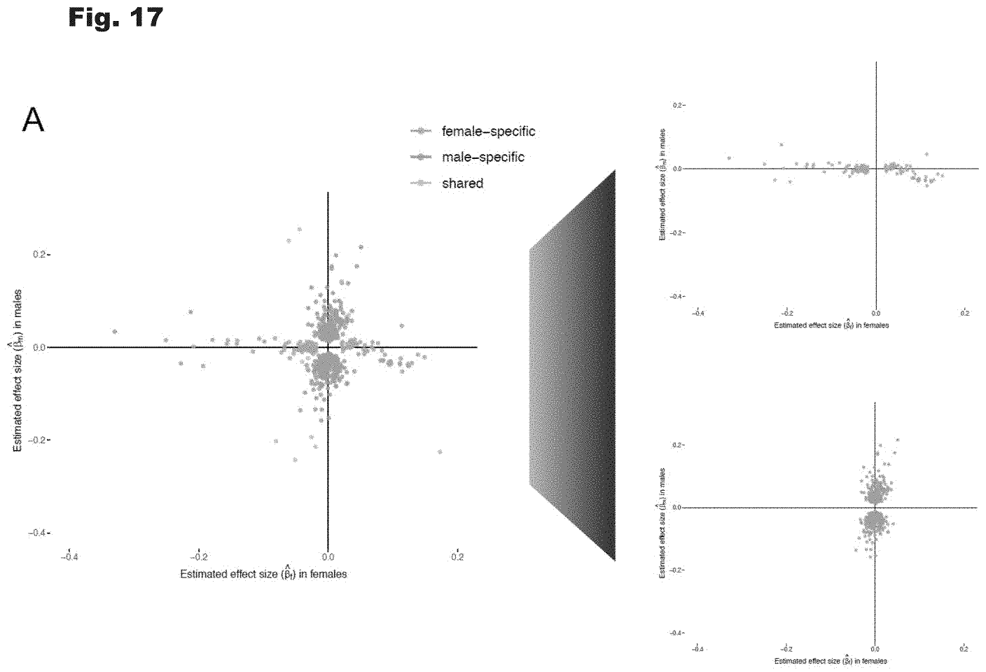

The four-component SEMM was applied to all 33 biomarkers to identify genetic variants with sex-specific and shared effects. In total, our analysis found 26,561 variants with effects on the traits of interest (Table 8 of FIG. 16). As expected, the majority (25,950) of these variants showed shared effects between sexes, and most traits had few or no sex-specific variants. However, 148 and 463 genetic variants with sex-specific effects in females and males were identified, the bulk of them associated with testosterone (80.4% and 96.1% respectively; see FIGS. 16A to 17). Of the testosterone variants, 54 male-specific, one female-specific, and one shared variant are located on the X chromosome, indicating enrichment of X chromosomal variants in male testosterone genetics. Additionally, using Tissue Specific Enrichment Analysis (X. Xu, et al., J. Neurosci. 34, 1420-1431 (2014), the disclosure of which is incorporated herein by reference), enrichment of liver genes was find in the genes proximal to male- but not female-specific variants (p=6.21×10⁻⁷ and p>0.1 respectively; FIG. 17).

Previous testosterone genetics studies have focused on males. In the current study, in addition to identifying male-specific variants in known testosterone-related genes (AR, JMJ1DC, and FAM9B), multiple female-specific variants with strong positive or negative effects on testosterone were also identified. In females, these include missense variants in STAG3 (rs149048452, β=−0.33 and p=8.97×10⁻⁹), a meiosis cohesion complex protein containing variants associated with premature ovarian failure, and POR (rs17853284, β=−0.23 and p=8.98×10⁻¹⁵), a cytochrome p450 oxidoreductase where deficiencies associated with amenorrhea, disordered steroidogenesis, and congenital adrenal hyperplasia. Many female-specific missense variants are located in genes associated with steroid hormone production (LIPE, POR, UGT2B7) or gamete formation (STAG3, MCM9, TSBP1, ZAN); although ZAN and TSBP1 encode the sperm zonadhesin protein and testis-expressed protein 1 respectively. These associations may help with understanding testosterone genetics in women.

### Mendelian Randomization of Sex-Specific Genetic Effects

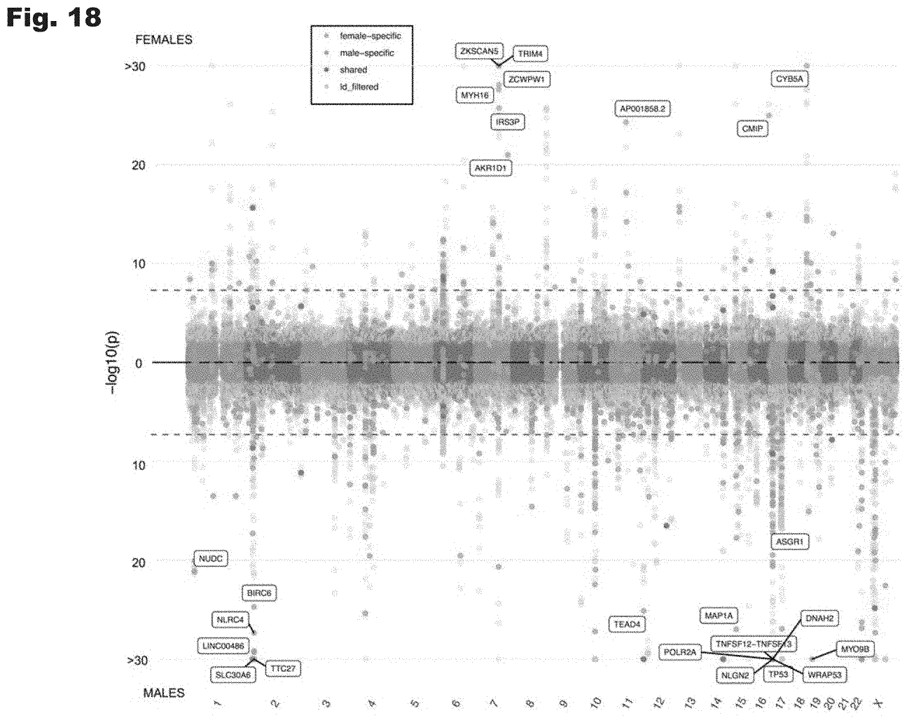

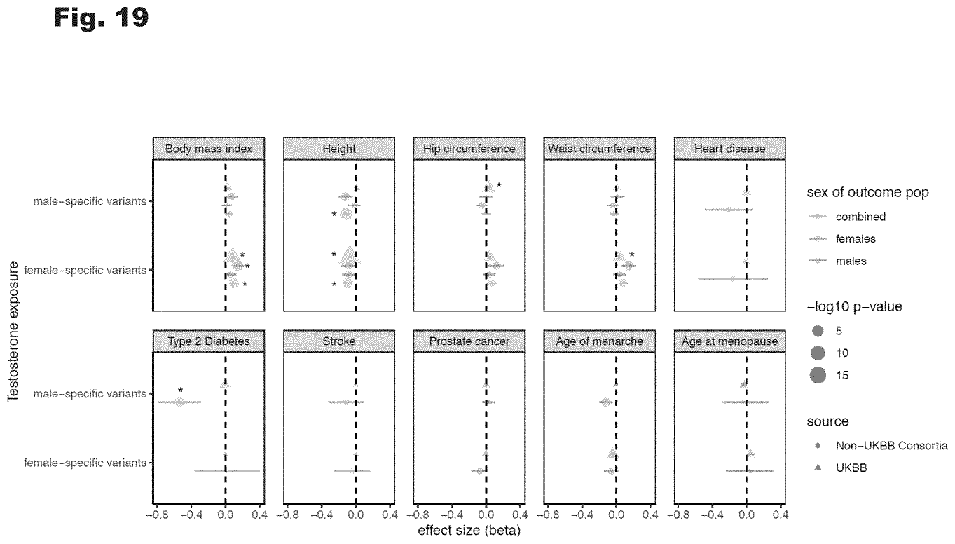

After identifying genetic variants with sex-specific effects on testosterone levels, Mendelian Randomization (MR) was used to examine whether these biomarkers are causally related to disease outcomes or other commonly measured traits. The intuition is that if a genetic variant is associated with differing levels of a biomarker, this provides a natural experiment, and one can examine whether the predicted variance in that biomarker based on the genetic variant is associated with the outcome variance, which indicates a causal effect. A total of ten outcomes were aggregated (Table 3 in FIG. 4), including anthropometric traits (height, BMI, waist circumference [WC], and hip circumference [HC]), disease outcomes (heart disease, stroke, and diabetes), and sex-specific traits (ages at menarche and menopause, prostate cancer), and used the Inverse-Variance Weighted (IVW) Method (J. Bowden, et al., Stat. Med. 36, 1783-1802 (2017), the disclosure of which is incorporated herein by reference). to assess the causal effects of the sex-specific variants identified in our analysis (FIG. 18). It was found that testosterone levels showed evidence of a causal association with BMI and WC using female-specific variants as instruments and HC using male-specific variants, with estimated effects consistent with higher testosterone increasing BMI, WC, and HC (p=1.3×10⁻¹², 1.1×10⁻⁴, 2.6×10⁻⁵; β=0.081, 0.04, 0.036; SE=0.011, 0.01, 0.0086 respectively, FIG. 19). A previous MR study (S. Lou, et al., BMJ 364, 1476 (2019), the disclosure of which is incorporated herein by reference). examined testosterone for causal effects on HC, WC, and BMI, but did not find evidence of an association; however, it is possible the current study was able to find these associations because the study used sex-specific genetic instruments. Both female and male variants showed evidence of a causal association with height (p=6.1×10⁻⁶ and 9.8×10⁻⁹), with higher testosterone associated with decreased height (β=−0.093 and −0.11, SE=0.021, 0.020). This is in contrast to evidence of a positive relationship between height and testosterone levels at a population level and in a previous MR study (S. Luo, et al., (2019), cited supra; D. J. Handelsman, et al., Eur. J. Endocrinol. 173, 809-817 (2015), the disclosure of which is incorporated herein by reference). For all of these associations, similar effects were observed in the UK Biobank and GIANT datasets, with MR Egger and IVW.

Male-specific testosterone levels show evidence of an association with type 2 diabetes (T2D) (p=3.1×10⁻⁵); higher testosterone is related to T2D risk reduction (β=−0.54, SE=0.13) using data from the combined DIAGRAM and MetaboChip study (A. P. Morris, et al., Nat. Genet. 44, 981-990 (2012), the disclosure of which is incorporated herein by reference). This association was found using the IVW method; MR Egger estimates indicate the relationship is in the reverse direction and is not significant (β=1.1, SE=0.61, p=0.10). Several longitudinal studies have shown that low levels of testosterone predict the later development of T2D or metabolic syndrome (L. Antonio, et al., J. Clin. Endocrinol. Metab. 100, 1396-1404 (2015), the disclosure of which is incorporated herein by reference).

### Sex-Specific Multivariate Polygenic Risk Prediction

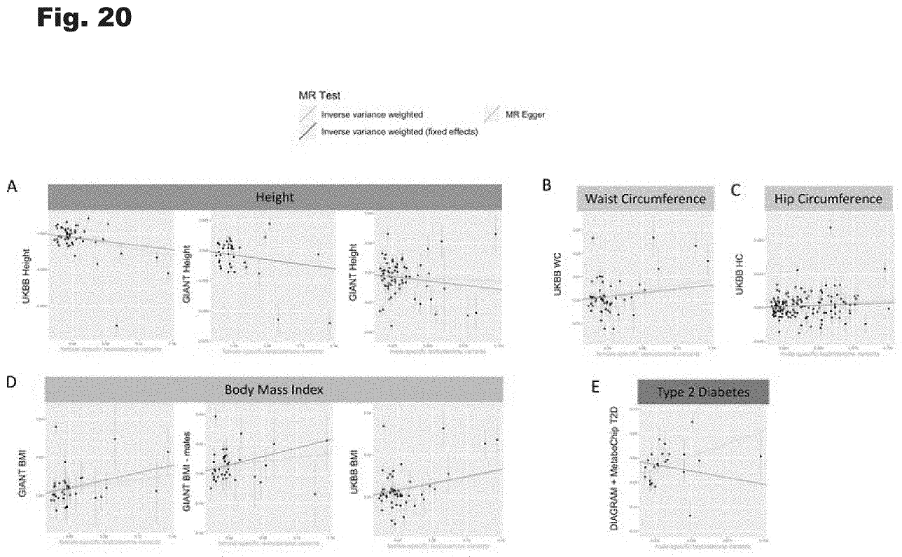

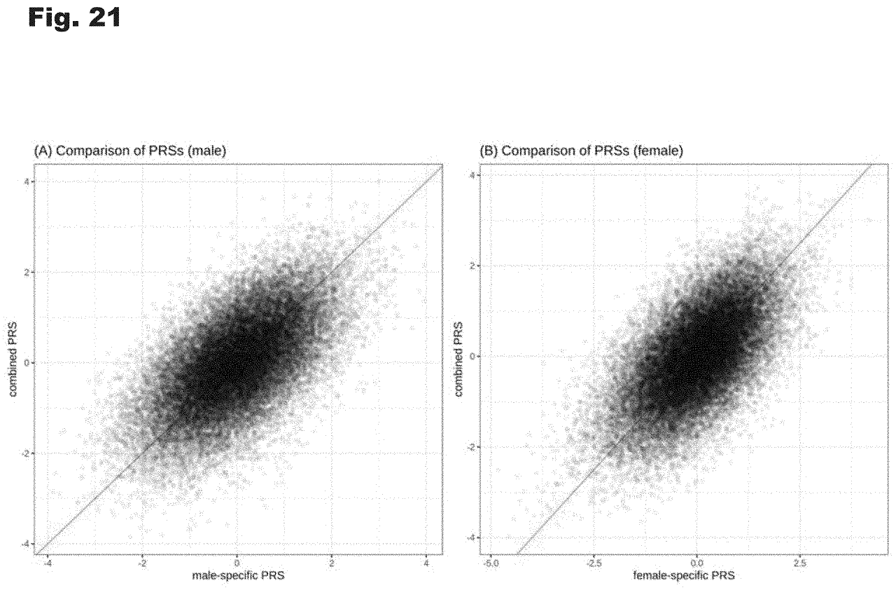

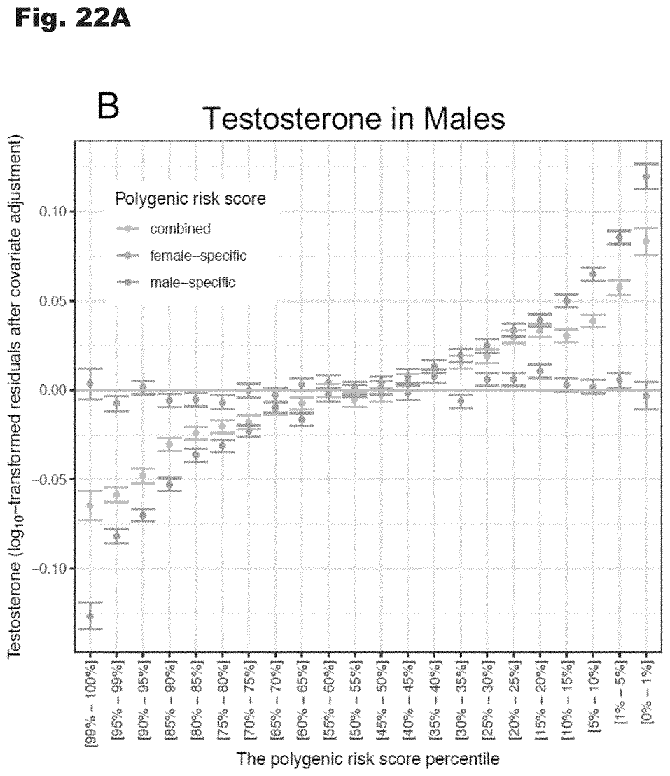

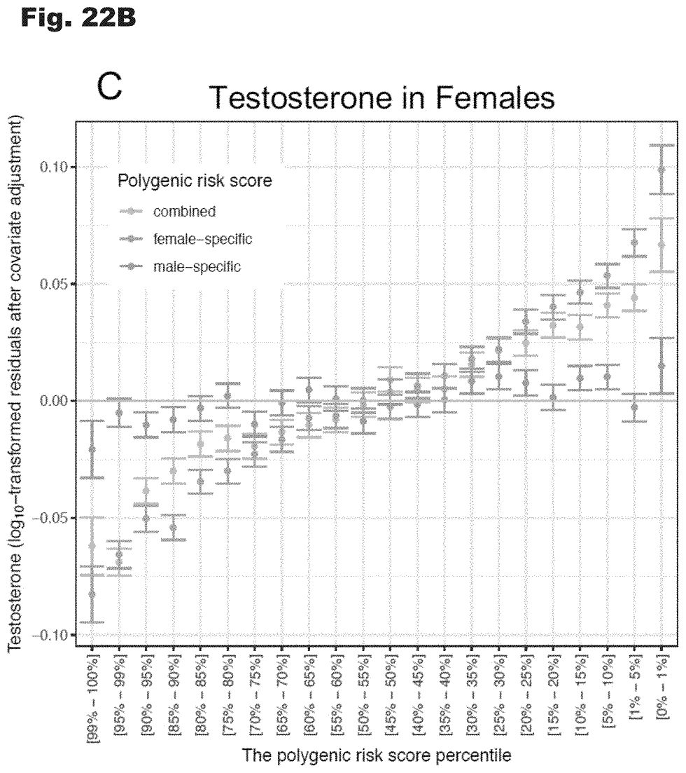

Motivated by the sex differences in testosterone genetics, it was tested whether sex-specific polygenic risk scores (PRS) would have better predict testosterone levels than a sex-combined model. Batch screening iterative lasso (BASIL) was applied to train multivariate penalized regression models for males and females (J. Qian, et al., bioRxiv 630079 (2019), the disclosure of which is incorporated herein by reference). While the two sex-specific and combined models are consistent on a held-out test set (p=0.59 and 0.60, p<2.2×10⁻¹⁶; FIG. 20), the sex-specific models have improved performance in the sexes they were trained on over the combined model, and low performance in the opposite sex (R²=0.31 vs 0.21vs 0.020 and 0.18 vs 0.13 vs 0.023 for male and female vs combined vs opposite sex). Overall, these results highlight the benefits of sex-specific polygenic prediction for testosterone (FIGS. 21A, 21B, and 22).

### Discussion

While there are reported sex differences in biomarker levels, the extent to which these differences are genetic is not known. To answer this question, the genetics of 33 biomarkers in UK Biobank males and females we studied using SEMM, a two and four-component Bayesian Mixture Model. SEMM has the benefit of both estimating the underlying genetic architecture and identifying genetic variants with shared and sex-specific effects. For the majority of the traits analyzed, a strong sex differences in genetic effects was not seen, which is expected and previously documented in the literature (S. Stringer, et al., Sci. Rep. 8, 18060 (2018), the disclosure of which is incorporated herein by reference). Namely, the genetics of these traits are shared (as indicated by genetic correlations close to one and similar heritabilities), and the traits have no or very few variants with sex-specific effects.

By contrast, little overlap was found between males and females in the genetics of testosterone levels. In addition to finding significant sex differences in genetic architecture, over five hundred genetic variants were identified with male- or female-specific effects. Because of the male-female differences in testosterone genetics, the subset of protein-altering variants and the tissue-specific expression patterns of genes with variants that have sex-specific genetic effects were examined. The protein-altering variants associated with female-specific effects testosterone include variants in genes associated with steroid hormone production and gamete production. Tissue-specific enrichment analysis reveals that the genes proximal to these sex-specific variants are enriched in liver in males but not females. It is hypothesized that this relationship between testosterone and liver disease may have different etiology in men and women. Additionally, sex-specific polygenic risk models were built, which showed improved predictive performance over a sex-combined model.

Mendelian Randomization was used to assess whether testosterone may be causally implicated in a broad range of diseases and phenotype measurements, and associations with BMI, WC, HC, height, and T2D were found. The relationships with BMI, WC, and HC are novel MR associations and it is possible that these were identified because of use of a novel set of genetic instruments. Further, most previous MR studies in testosterone excluded women. This analysis shows a decreasing effect of testosterone on height, which is surprising, as previous studies indicated that higher testosterone is associated with higher stature. However, testosterone is sometimes used as a therapy for tall males with delayed puberty, and results in accelerated initial growth but overall stunting of stature (M. Zachmann, et al., J. Pediatr. 88, 116-123 (1976), the disclosure of which is incorporated herein by reference). Further work is required to understand this association. Finally, the potential causal relationship with T2D supports the hypothesis that testosterone treatment for reducing diabetes risk in men may be a worthwhile approach, and matches the MR recent findings of Ruth et al. (K. S. Ruth, et al., Nat. Med. 26, 252-258 (2020), the disclosure of which is incorporated herein by reference), that higher testosterone levels reduce T2D risk in men and increase risk in women. While sex-separated T2D data was not used in the current MR analysis, the associations between T2D and testosterone levels were only found with male-specific testosterone variants as instruments, which is consistent with this effect.

Testosterone is frequently thought of as a male sex hormone because of its higher levels in men and involvement in the development of the male reproductive tract and secondary sex characteristics. However, females also produce testosterone, albeit at lower levels, and elevated testosterone is associated with polycystic ovarian syndrome and metabolic disorders (R. Haring, et al., Eur. J. Cardiovasc. Prev. Rehabil. 18, 86-96 (2011); and J. J. Kim, et al., Aliment Pharmacol Ther. 45, 1403-1412 (2017), the disclosures of which are incorporated herein by reference). Previous work examining the genetics of testosterone in females did not find associations and previous Mendelian Randomization studies have been limited by the lack of known testosterone variants in women (S. Lou, et al., (2019), cited supra; J. Prescott, et al., PLoS ONE 7, e37815 (2012), the disclosure of which is incorporated herein by reference). The current analysis expands on and addresses this by using a larger population, carefully adjusted biomarkers, and our SEMM method to identify variants. The results demonstrate that the genetics of testosterone levels is complex and highly polygenic in both males and females. Further, the current work highlights the importance of also examining female variability in testosterone levels, and of considering sex as a variable.

### Supplementary Methods

### White British Ancestry Definition

This definition was based on the following five criteria reported by the UK Biobank in the file “ukb_sqc_v2.txt”:
1. used to compute principal components (“used_in_pca_calculation” column)
2. not marked as outliers for heterozygosity and missing rates (“het_missing_outliers” column)
3. do not show putative sex chromosome aneuploidy (“putative_sex_chromo-some_aneuploidy” column)
4. have at most 10 putative third-degree relatives (“excess_relatives” column).
5. White British ancestry (“in_white_British_ancestry_subset” column)

A subset of individuals was subsequently focused on with non-missing values for covariates and biomarkers as described below. Unrelated individuals were defined with criteria 1 and 4.

### Derivation of Anthropometric Traits

Four anthropometric traits were derived: arm-fat-ratio, leg-fat-ratio, trunk-fat-ratio, and waist-hip-ratio (WHR). WHR was calculated by taking the ratio of waist circumference (ID:48) to hip circumference (ID:49). Fat ratio traits were calculated as described in E. A. Khramtsova, et al. (Nat. Rev. Genet. 20, 173-190 (2019), the disclosure of which is incorporated herein by reference).using impedance measures. Briefly, we took the fat mass for each body area: trunk (ID:23128), arm (ID:23124, 23120), or leg (ID:23116, 23112)—for arm and leg we summed right and left together—and divided by the total fat mass (ID:23100). For each of the anthropometric traits, individuals missing data or with values outside six standard deviations of the mean were removed.

### Selection of Biomarker Traits

Biomarker traits included in the analysis were previously examined in Sinnott-Armstrong et al. ((2019), cited supra), consisting of blood biochemistry assays (28 total) and urinalysis (4 total) and two derived blood biomarkers (Non-albumin protein, eGFR). Cholesterol, Apolipoprotein B, and LDL were adjusted by statins. Two of the blood biochemistry assays (Oestradiol, Rheumatoid Factor) and one urine assay (Microalbumin in urine) were excluded because they had a large fraction of levels below the reported range. Covariate correction of biomarkers was performed as reported previously (M. Rask Andersen, et al., (2019), cited supra) using sex-separated data from White British individuals. For testosterone analysis, individuals taking the following testosterone-related drugs were removed: methyltestosterone, finasteride, dutasteride, testosterone, mesterolone, and cyproterone.

### Menopause Phenotype Definition

Pre-menopause was defined as self-reported “reached menopause” (field ID: 2724) and less than 60 years old and post-menopause as at least 2 years post menopause (ID:3581) and had menopause after age 40 (to exclude premature menopause). People with missing or “unsure” answers and individuals who may have gone through surgical menopause were excluded (oophorectomy ID:2834 or hysterectomy ID:3591 or 2724). In the White British population, this resulted in 35,999 pre- and 91,462 post-menopausal women (53,573 excluded).

### Model Descriptions

All models were constructed and fit in STAN (version 2.17.2)(Q. Ostrom, et al., bioRxiv 229112 (2017), the disclosure of which is incorporated herein by reference). All other code was written in R version 3.5.1.

### Two Component SEMM—Estimating Variance-Covariance Matrix for Sex Divided Data

A two-component model was used to estimate genetic parameters (genetic correlation between males and females) and calculate heritability. Briefly, a two-dimensional vector of summary statistics was construct for each variant i, using the male- and female-sex divided summary stats:

$${\hat{β}}_{i} = [ \begin{matrix}{\hat{β}}_{i , f} \\ {\hat{β}}_{i , m}\end{matrix} ] , {\hat{SE}}_{i}^{2} = [ \begin{matrix}{\hat{SE}}_{i , f}^{2} \\ {\hat{SE}}_{i , m}^{2}\end{matrix} ]$$

Each variant is modeled as belonging to either an “effect” component or a “no effect” component based on the summary statistics. The effect component contains a variance-covariance matrix that describes how the male- and female-effects covary. The set of all variants was used to estimate the parameters of these components.

To formulate this, a two-component mixture model consisting of a point mass centered at zero (“no effect” or null component) and a multivariate normal distribution (“effect” or non-null component) was used. The components are described below:

$${M}_{0} = [ \begin{matrix}{\hat{β}}_{f} \\ {\hat{β}}_{m}\end{matrix} ] \sim N ( 0 , [ \begin{matrix}{\hat{SE}}_{f}^{2} & 0 \\ 0 & {\hat{SE}}_{m}^{2}\end{matrix} ] )$$

$${M}_{1} = [ \begin{matrix}{\hat{β}}_{f} \\ {\hat{β}}_{m}\end{matrix} ] \sim N ( 0 , [ \begin{matrix}{\hat{SE}}_{f}^{2} & 0 \\ 0 & {\hat{SE}}_{m}^{2}\end{matrix} ] + {Σ}_{g} )$$

where Σ_{g} is the genetic variance-covariance matrix for the non-null component. Σ_{g} consists of the male and female-specific variances σₘ² and σ_{f}², and a scaling factor ρ:

$${Σ}_{g} = [ \begin{matrix}{σ}_{f}^{2} & {ρσ}_{f} {σ}_{m} \\ {ρσ}_{f} {σ}_{m} & {σ}_{2}^{m}\end{matrix} ]$$

The likelihood across all variants is then formulated as:

$$L = {\sum }_{i} log ( {π}_{0} N ( [ \begin{matrix}{\hat{β}}_{i , f} \\ {\hat{β}}_{i , m}\end{matrix} ] ; 0 , [ \begin{matrix}{\hat{SE}}_{f}^{2} & 0 \\ 0 & {\hat{SE}}_{m}^{2}\end{matrix} ] ) + {π}_{1} N ( [ \begin{matrix}{\hat{β}}_{i , f} \\ {\hat{β}}_{i , m}\end{matrix} ] ; 0 , [ \begin{matrix}{\hat{SE}}_{f}^{2} & 0 \\ 0 & {\hat{SE}}_{m}^{2}\end{matrix} ] + [ \begin{matrix}{σ}_{f}^{2} & {ρσ}_{f} {σ}_{m} \\ {ρσ}_{f} {σ}_{m} & {σ}_{m}^{2}\end{matrix} ] ) )$$

where i is the ith variant and π₀ is the proportion in the null component and π₁ is the proportion in the non-null component.

The model priors were set as follows:
π˜Beta(1, 1)
τ˜Half Cauchy(0, 2.5)
Ω˜LKJCorr(2)
Σ_{g}=TΩτ

π is a two-dimensional vector containing π₀ and π₁. The parameters Ω and τ to force Σ_{g} were used to be a 2-by-2 matrix variance-covariance matrix. A Beta distribution centered at (1,1) was used for π in order to not favor assignment to either component. Priors were chosen for Σ_{g} based on suggestions from the STAN Manual (N. Sinnott-Armstrong, et al., (2019), cited supra). A half-Cauchy distribution with a small scale was chosen for T to be a weakly informative scaling prior and the LKJ correlation distribution was chosen as a prior for Ω. The LKJ distribution is the uniform for v=1, and as v increases for v>1 the distribution shows less correlation between components and increasingly concentrates around the unit matrix, while for v<1 favors more correlation. Additionally, a variety of prior parameters were tried, which did not affect the resulting estimates of π and Σ_{g} (see Comparison of prior parameters below).

### Four Component SEMM—Identifying Sex-Specific Genetic Variants

A mixture model with four components: (0) no effect, (1) female-specific effect, (2) male-specific effect, and (3) effects in both sexes was formulated in order to identify variants with sex-specific effects. The idea is that each variant belongs to a particular component indicating its effect on the phenotype. The variant summary statistics were used to formulate the distributions for each of the components.

Let β be the true effect and k refers to the component. Here β₍₁₎, β₍₂₎, β₍₃₎, β₍₄₎≠0:

k=0, βₘ=β_{f}=0 No effect

k==1, βₘ=β₍₁₎, βₘ=0 Female-specific effect

k=2, β_{f}=0, βₘ=β₍₂₎Male-specific effect

k=3, βₘ=β₍₃₎, β_{f}=β₍₄₎ Effects in both sexes

We formulate each of the four components k=j as follows:

$$[ \begin{matrix}{\hat{β}}_{f} \\ {\hat{β}}_{m}\end{matrix} ] \sim N ( 0 , [ \begin{matrix}{\hat{SE}}_{f}^{2} & 0 \\ 0 & {\hat{SE}}_{m}^{2}\end{matrix} ] + {Σ}_{j} )$$

where:

$${Σ}_{0} = [ \begin{matrix}0 & 0 \\ 0 & 0\end{matrix} ] , {Σ}_{1} = [ \begin{matrix}{σ}_{1 , f}^{2} & 0 \\ 0 & 0\end{matrix} ] , \\ {Σ}_{2} = [ \begin{matrix}0 & 0 \\ {σ}_{2 , m}^{2} & 0\end{matrix} ] , {Σ}_{4} = [ \begin{matrix}{σ}_{3 , f}^{2} & 0 \\ 0 & {σ}_{3 , m}^{2}\end{matrix} ]$$

Here, π, the proportion in each component, and σ_{1,f}², σ_{2,m}², σ_{3,m}², σ_{3,f}² the non-zero variances for each component were estimated. A Dirichlet prior centered at (1,1,1,1) was chosen for π in order to not favor any component at the start. InverseGamma(1,1) priors were used to cover a range of values expected for the nonzero variances σ².

### Assignment of Variants to Components

The STAN programming language does not allow for assignment samples to components in mixture models; however, this assignment is needed in order to identify which variants have sex-specific effects (e.g. are assigned to sex-specific components). Therefore estimated parameters, were used from the model to assign variants to components. Let γᵢ be the component to which variant i. is assigned. The parameter p_{i,k} is the probability of variant i in component k (i.e. γᵢ=k), and this is modeled as a multinomial distribution with probability pᵢ. is the vector of four p_{i,k} for a particular variant. And the probability π_{i,k} is estimated based on the summary statistics for that variant i:

$${\hat{β}}_{i} = [ \begin{matrix}{\hat{β}}_{i , f} \\ {\hat{β}}_{i , m}\end{matrix} ] , {\hat{SE}}_{i}^{2} = [ \begin{matrix}{\hat{SE}}_{i , f}^{2} \\ {\hat{SE}}_{i , m}^{2}\end{matrix} ]$$

and estimated parameters. This is formulated below:

$${p}_{i , k} = \frac{{π}_{k} N ( {\hat{β}}_{i} : 0 , {\hat{SE}}_{i} + {\sum }_{k} )}{{\sum }_{k} {π}_{k} N ( {\hat{β}}_{i} : 0 , {\hat{SE}}_{i} + {\sum }_{k} )}$$

Variants were assigned to a non-null component if they had a posterior probability p_{i,k}>0.8 of being in that component; otherwise, they were assigned to the null component.

### False Discovery Rate (FDR) Calculation

To estimate the FDR for the variants associated with a trait, we first assigned the variants to one of the four components based on a set posterior probability cutoff, as described above. For each of the non-null components (e.g. k=1, 2, 3, not k=0), the null (p₀) posterior probabilities was averaged across all variants assigned to that component. Results were calculated for components with more than 5 assigned variants because parameter estimates for very small components are often inflated (this threshold of 5 variants could have been adjusted to report more or fewer FDR estimates). The FDR was calculated for a range of posterior probability cutoffs (0.5-0.9) (see Tables 4A to 4D of FIGS. 7A to 7D, and FIG. 8 for the FDR estimates at each cutoff). In order to approximate an FDR≤0.05, a posterior cutoff of 0.8 was selected because it is the lowest cutoff where the interquartile range of our calculated FDR values lies under an FDR value of 0.05.

### Computation of Genetic Correlation and Heritability

Genetic correlations were estimated by Ω from the fit, the 2.5 to 97.5 percentile interval was given from the STAN fit of the parameter. Estimates for π and Σ are also extracted using the median value (50%) of the STAN fit for those parameters. For each trait, the sex-specific heritability in males or females (x=m or f) was calculated by first assigning variants to components based on their posterior probability. Then using all variants assigned to the non-null component, heritability was estimated by the following equation, where n₁ is the number of variants in the non-null component and π₁ is the fraction in the non-null component.

$${h}_{x} = \frac{{n}_{1} {π}_{1} {σ}_{x}^{2}}{{n}_{1} {π}_{1} {σ}_{x}^{2} + {Σ}_{i}^{{n}_{1}} {\hat{SE}}_{i , x}^{2}}$$

To create a 2.5 to 97.5 percentile interval, the posterior probability for each variant and the overall heritability was calculated as described above using the π and Σ estimates from each of the post-warmup draws. These estimates were then ordered, and the 2.5 and 97.5 percentile are reported.

### Comparison of Prior Parameters

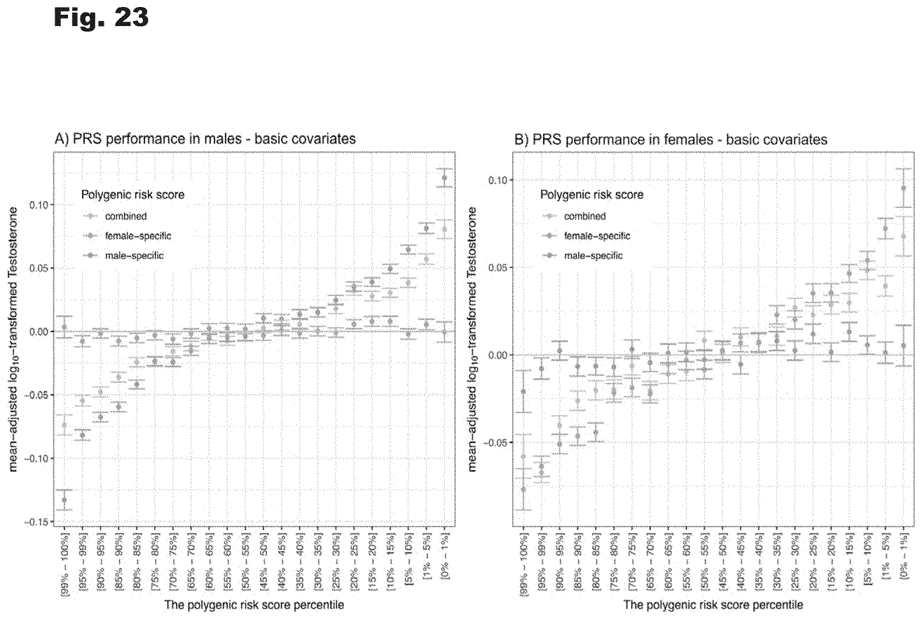

A variety of different parameters were tried for the priors. For model 1, results showed little variation in the parameter estimates for the fraction in each component, the variance-covariance matrix, and the genetic correlation (FIG. 13). The expected log-predictive density was calculated with leave-one-out cross-validation (ELPD-LOO) for each of these prior parameterizations using the loo R package (B. Bulik-Sullivan (2015), cited supra; and W. G. Hill (1978), cited supra), and the difference (ELPD-DIFF) between these parameterizations indicate that one is not better than any of the others. All loo calculations were performed on the same randomly selected subset of 20,000 variants. For model 2, comparison across parameterizations indicated that the model 2 estimates did not appear to be affected by the choice of dirichlet prior; however, estimates were sensitive to the choice of the inverse gamma parameters (FIG. 23), this was also shown in the estimated ELPD-LOO, which was highest for an inverse_gamma(10,1) prior, indicating that this choice of prior fit the data best.

### Assessment of Convergence

All models were run with 4 chains with 1000 warm up and 2000 total iterations. Convergence was assessed by examination of Rhat and n_eff. Rhat measures the consistency of chains, an Rhat close to 1 indicates the chains are consistent. For all parameters of both M1 and M2 testosterone model fits, Rhat was in the range 0.999 to 1.007. n_eff captures the effective sample size, it is concerning if it is very small. For all parameters in the M1 and M2 models, n_eff was greater than 1555 (median 2013 for M1, 3688 for M2). All fits were also checked for divergence and no model fits contained post-warmup divergences.

### Tissue-Specific Enrichment Analysis (Additional Description)

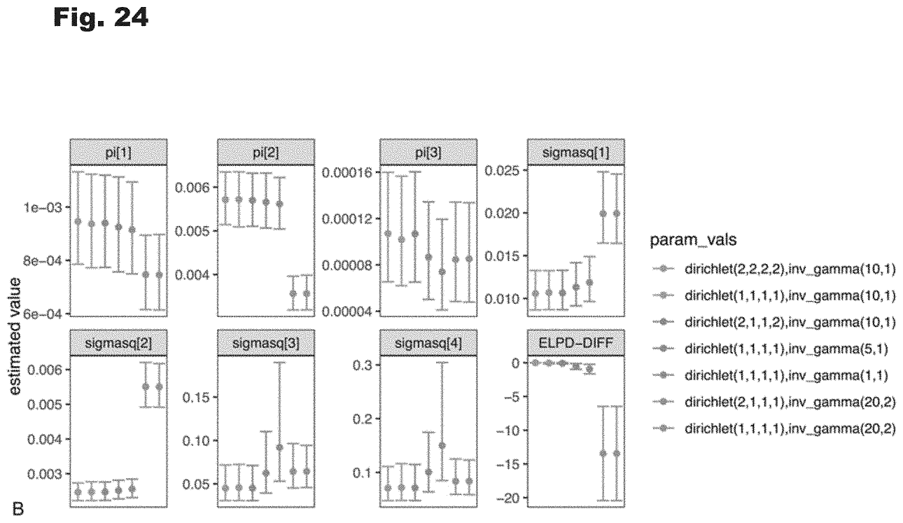

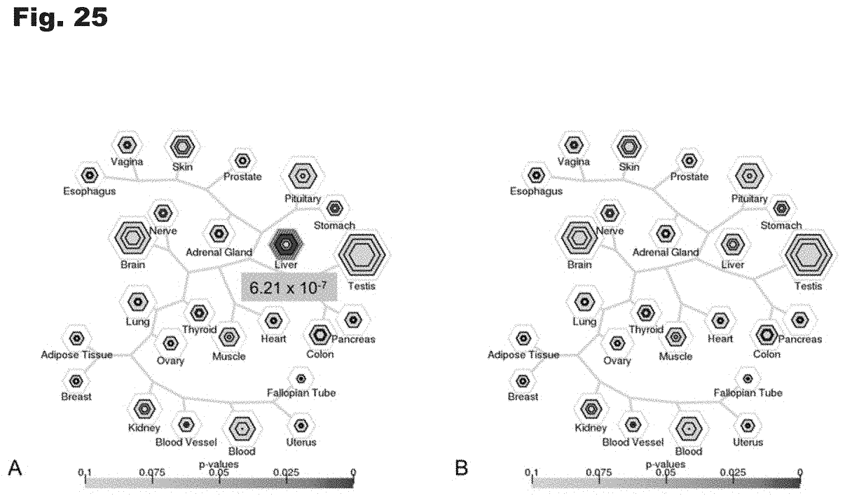

TSEA uses published RNA-seq data GTEx data, and calculates the enrichment of a list of genes in the genes specific to each of twenty-five tissues. Briefly, data from 45 tissues (including sub-tissues) was grouped into 25 “whole-tissue” types by averaging the gene level read counts. A specificity index statistic (G. Ni., et al. (2018), cited supra) was used to identify enriched genes for each of these tissues at three different thresholds (0.0001, 0.001, 0.01), where genes at lower thresholds are more specific to that tissue. A Fisher's Exact test is then used to calculate a p-value for the enrichment of a set of genes in that set; this is Benjamini-Hochberg corrected to account for multiple tests. TSEA's list of enriched genes for each tissue was calculated using sex-combined data, and whether the genes proximal to either the male or female-specific testosterone variants were examined for overrepresentation in the enriched gene lists. It is important to note that the TSEA tissue-specific profiles are not sex-specific, and future work may involve assessment of sex-specific variants using sex-divided expression profiles. See FIG. 24.

### PRS Training and Assessment (Additional Description)

Batch screening iterative lasso (BASIL) implemented in the R snpnet package was applied to independently train sex-specific PRS models for males (training n=100,913; validation n=14,594) and females (training n=99,564; validation n=14,049), and a baseline “combined” model consisting of both datasets. The validation set was used to select the optimal lambda value for sparsity. The input variants to the models were a combined genotype dataset consisting of array-genotyped variants, copy number variants, and HLA allelotypes. Non-zero regression coefficients (BETAs) were extracted from the optimal model and computed PRS using the -score function implemented in plink version 2.00a2LM (26 Aug. 2019). To evaluate the performance of “sex-specific” PRS or the “combined” PRS, individuals in the test set (males n=28,601; females n=28,640; combined n=57,241) were stratified based on the PRS bins and computed the mean value and standard error for each bin. The standard error was computed by dividing the sample standard deviation by the square root of the sample size in the bin. The consistency of sex-specific PRS and the combined PRS were compared and evaluated using their Spearman's rank correlation (ρ) (plots for comparison are located in FIG. 20). To address the setting where not all covariates are available, covariate adjustment was also performed using only age and genotype ancestry PCs 1-10 with similar results (FIG. 22).

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A method to determine hormone level based on genetic data, comprising:
obtaining or having obtained genetic sequencing data of an individual;
identifying or having identified variants within the sequencing result of the individual;
determining or having determined hormone level based on the identified variants, wherein the expected level of hormone is determined utilizing a linear model and the identified variants.

2. The method of claim 1, wherein the hormone is testosterone, estrogen, oxytocin, vasopressin, or progesterone.

3. The method of claim 1, wherein the genetic sequencing data is a whole genome, a partial genome, or an exome.

4. The method of claim 1, wherein the genetic sequencing data is a set of loci that have been found to be important to determine the level of the hormone.

5. The method of claim 1, wherein the genetic sequence data is derived from a biopsy of the individual.

6. The method of claim 1, wherein the trained linear model provides a polygenic risk score indicating the individual's level of hormone.

7. The method of claim 1, wherein the model is a multivariate penalized regression.

8. The method of claim 1, wherein a batch screening iterative lasso (BASIL) is used to train the linear model.

9. The method of claim 1, wherein the linear model is trained utilizing genetic variant data derived from a collection of people, each person of the collection having the same sex as the individual.

10. The method of claim 1, wherein the linear model is trained utilizing genetic variant data derived from a collection of people, where the collection of people includes males and females.

11. The method of claim 1, further comprising performing a clinical assessment based on the hormone level determination, wherein the clinical assessment is a blood test, medical imaging, or clinical test.

12. The method of claim 1, wherein the clinical test is to detect diabetes, insulin resistance, dyslipidemia, hypertension, polycystic ovarian syndrome (PCOS) disease, virilization, amenorrhea, hirsutism, hypothyroidism, hypopituitarism, delayed puberty, precocious puberty, premenstrual dysphoric disorder (PMDD), erectile dysfunction, female sexual dysfunction, hypogonadism, or infertility.

13. The method of claim 1, wherein the individual is determined to have a high level of the hormone, and the method further comprising treating the individual to decrease the level of the hormone.

14. The method of claim 13, wherein the hormone is testosterone and the treatment is administration of a glucocorticosteroid, metformin, an oral contraceptive, or spironolactone.

15. The method of claim 13, wherein the hormone is testosterone and the individual is female; wherein the individual is administered an agonist of LIPE, FBXO15, or DCAF12.

16. The method of claim 13, wherein the hormone is testosterone and the individual is female; wherein the individual is administered an antagonist of TRMU, SLC22A11, ZAN, STAG3, CYP3A43, POR, MCM9, MFSD9, or PER3.

17. The method of claim 13, wherein the hormone is testosterone and the individual is male; wherein the individual is administered an antagonist of ANXA9, BCO2, EML4, GAB1, HNF4A, JMJD1C, MACF1, METTL21A, NF1, P2RX7, PP1P5K1, QARS, RPTN, or SLCO1A2.

18. The method of claim 1, wherein the individual is determined to have a low level of the hormone, and the method further comprising treating the individual to increase the level of the hormone.

19. The method of claim 18, wherein the hormone is testosterone and the treatment is administration of testosterone replacement therapy (TRT), methyltestosterone, finasteride, mesterolone, cyproterone, vitamin D, or caffeine.

20. The method of claim 18, wherein the hormone is testosterone and the individual is male; wherein the individual is administered an antagonist of ACSL5, DNAH2, HGFAC, KIF27, KLHL38, LRRC14, MARS, MSH5, PPP2R3A, PRMT6, SERPINA1, SERPINF2, SLC17A2, SLCO1A2, SOS2, SPAST, SPDL1, TGM7, USP8, YY1AP1, ZBTB10, or ZCCHC6.
